SEQUENCE LISTING (21 sequences)

SEQ ID NO: 1 - DNA, 12 bp, Homo sapiens
cgcgcgcgcgcg

SEQ ID NO: 2 - DNA, 12 bp, Homo sapiens
gggcccgggccc

SEQ ID NO: 3 - DNA, 12 bp, Homo sapiens
ggtggcggtggc

SEQ ID NO: 4 - DNA, 888 bp, Homo sapiens
  misc_feature (321)..(327): hexanucleotide repeat that is repeated n-times
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcggg

SEQ ID NO: 5 - protein, 31 aa, Homo sapiens
WSGRARGRARGGAAVAVPAPAAAEAQAVASG

SEQ ID NO: 6 - protein, 34 aa, Homo sapiens
GRGRGGPGGGPGAGLRLRCLRPRRRRRRRWRVGE

SEQ ID NO: 7 - protein, 54 aa, Homo sapiens
GVVGAGPGAGPGRGCGCGACARGGGGAGGGEWVSEEAASWRVAVWGSAAGKRRG

SEQ ID NO: 8 - protein, 23 aa, Homo sapiens
SARLLSSRACYRLRLFPSLFSSG

SEQ ID NO: 9 - protein, 6 aa, Homo sapiens
PLARDS

SEQ ID NO: 10 - protein, 10 aa, Homo sapiens
QALELRSRAL

SEQ ID NO: 11 - protein, 122 aa, Homo sapiens
GRESKEEARSPSLVPAPPPPPPPPGSPGPGCRQFHQSLEAKARHPASVREMRGKVKMRRALRRAPASTRASSRQPNPKQPPARMPPPHSPTRHRLRLRRRGRRHRNRSPAPGPPPGPPRPRP

SEQ ID NO: 12 - protein, 26 aa, Homo sapiens
RLTRRKQGGKQPQPVASSGTQESRAR

SEQ ID NO: 13 - protein, 59 aa, Homo sapiens
GEPPLLPAPLPGSRTPNSHPPGCRLLTHPLATACASAAAGAGTATAAPPRARPRARPDH

SEQ ID NO: 14 - protein, 160 aa, Homo sapiens
SPRRQGPSRVPSEPRLGPQKPRAAHPPAFPQARPLSTRGSLFSSPQRQRSQRVPGKETARVLRAGKQARMQAIPPVARGESPTPSFGQRNERESKNASSSEESPRFYPRLFPAAEPQTATRQDAASSLTHSPPPAPPPPRAQAPQPQPRPGPAPGPAPTT

SEQ ID NO: 15 - protein, 47 aa, Homo sapiens
  MISC_FEATURE (13)..(14): amino acids are repeated m-times
QALELRSRALGAGAGAWSGRARGRARGGAAVAVPAPAAAEAQAVASG

SEQ ID NO: 16 - protein, 40 aa, Homo sapiens
  MISC_FEATURE (2)..(3): amino acids are repeated o-times
GPGPGPGRGRGGPGGGPGAGLRLRCLRPRRRRRRRWRVGE

SEQ ID NO: 17 - protein, 128 aa, Homo sapiens
  MISC_FEATURE (122)..(123): amino acids are repeated o-times
GRESKEEARSPSLVPAPPPPPPPPGSPGPGCRQFHQSLEAKARHPASVREMRGKVKMRRALRRAPASTRASSRQPNPKQPPARMPPPHSPTRHRLRLRRRGRRHRNRSPAPGPPPGPPRPRPGPGPGP

SEQ ID NO: 18 - protein, 87 aa, Homo sapiens
  MISC_FEATURE (29)..(30): amino acids repeated p-times
RLTRRKQGGKQPQPVASSGTQESRARGRGRPGRGVVGAGPGAGPGRGCGCGACARGGGGAGGGEWVSEEAASWRVAVWGSAAGKRRG

SEQ ID NO: 19 - protein, 88 aa, Homo sapiens
  MISC_FEATURE (62)..(63): amino acids repeated q-times
GEPPLLPAPLPGSRTPNSHPPGCRLLTHPLATACASAAAGAGTATAAPPRARPRARPDHAPAPAPSARLLSSRACYRLRLFPSLFSSG

SEQ ID NO: 20 - protein, 196 aa, Homo sapiens
  MISC_FEATURE (186)..(187): amino acids are repeated r-times
SPRRQGPSRVPSEPRLGPQKPRAAHPPAFPQARPLSTRGSLFSSPQRQRSQRVPGKETARVLRAGKQGRGQIPIPCPCAAAAAAAAGKPGARMQAIPPVARGESPTPSFGQRNERESKNASSSEESPRFYPRLFPAAEPQTATRQDAASSLTHSPPPAPPPPRAQAPQPQPRPGPAPGPAPTTPRPRRPRPLARDS

SEQ ID NO: 21 - protein, 21 aa, Homo sapiens
  MISC_FEATURE (2)..(2): amino acid is NLE Norleucine
GXGAGAGAGAGAGAGAGAGAG

(19) United States
(12) Patent Application Publication
(10) Pub. No.: US 2022/0202935 A1
(43) Pub. Date: Jun. 30, 2022

(54) Immunogen for Preventing or Treating Familial Frontotemporal Dementia (FTD) and/or Amyotrophic Lateral Sclerosis (ALS)

(71) Applicant: DEUTSCHES ZENTRUM FÜR NEURODEGENRATIVE ERKRANKUNGEN E.V.(DZNE), Bonn (DE)

(72) Inventors: Dieter Edbauer, Germering (DE); Qihui Zhou, Munich (DE); Nikola Marelijic, Munich (DE)

(21) Appl. No.: 17/606,851

(22) PCT Filed: May 4, 2020
(86) PCT No.: PCT/EP2020/062343

§ 371 Date: Oct. 27, 2021

(30) Foreign Application Priority Data
May 2, 2019 (EP) 19172392.3

(51) Int. Cl. A61K 39/395; A61P 25/28

(52) U.S. Cl. CPC A61K 39/39533; A61K 2039/6093; A61P 25/28

(57) ABSTRACT

The present invention relates to an immunogen for use in preventing or treating familial frontotemporal dementia (FTD), amyotrophic lateral sclerosis (ALS) and/or amyotrophic lateral sclerosis-frontotemporal dementia (ALS-FTD) in patients with C9orf72 repeat expansion. The immunogen is comprising or consisting of a polypeptide consisting of dipeptide-repeats with a sequence selected from the group consisting of (Gly-Ala)a, (Gly-Pro)a, (Gly-Arg)a, (Pro-Ala)a and (Pro-Arg)a, wherein a is an integer of 4 to 25.

The present invention relates to an immunogen for use in preventing or treating familial frontotemporal dementia (FTD) and/or amyotrophic lateral sclerosis (ALS) comprising or consisting of a polypeptide consisting of dipeptide-repeats as well as an immunogenic composition comprising the immunogen and its use. Furthermore, a kit comprising the immunogen and a method of treatment is disclosed.

### BACKGROUND OF THE INVENTION

Neurodegenerative disorders are generally classified by characteristic protein deposits. Moreover, in a number of neurodegenerative diseases rare genetic mutations causing inherited variants of the disease were associated with the genes encoding the aggregating/deposited proteins, their precursors or their modulating enzymes. Frontotemporal lobar dementia (FTD) and amyotrophic lateral sclerosis (ALS) are the extreme ends of a spectrum of overlapping neurodegenerative disorders variably associated with dementia, personality changes, language abnormalities and progressive muscle weakness (Josephs et al., 2011; Mackenzie et al., 2010; Rademakers et al., 2012). Research into ALS and FTD was dramatically accelerated by the identification of the RNA/DNA binding protein TDP-43 (Tar DNA binding protein of 43 kDa) as an abundant deposited protein (Arai et al., 2011; Neumann et al., 2006) and by the discovery that mutations in TARDBP cause familial variants of both diseases (Benajiba et al., 2009; Sreedharan et al., 2008). The majority of ALS and FTD cases show cytoplasmic inclusions that are strongly positive for phosphorylated TDP-43, although TDP-43 is normally localized mainly in the nucleus. These findings also helped to develop the concept that ALS and FTD are multisystem disorders with overlapping clinical and pathological characteristics and similar functional and genetic causes (Rademakers et al., 2012; Sieben et al., 2012) and which are therefore classified as FTD-TDP, FTD/ALS-TDP or ALS-TDP. Besides TDP-43, and the long known SOD1 (super oxide dismutase 1) gene, a number of other ALS and/or FTD related genes/risk factors were discovered including FUS (Fused in Sarcoma), OPTN (optineurin), Ataxin 2, Chmp2B, VCP (Valosin containing protein), TMEM106B, GRN (Progranulin), PFN (Profilin) and the C9orf72 gene. Pathological repeat expansions in C9orf72 have been found in about 40% of familial ALS patients and 20% of familial FTD. Despite large heterogeneity in different populations expansion of a GGGGCC hexanucleotide repeat upstream of the C9orf72 coding region is the most common known cause of familial frontotemporal dementia (FTD) and amyotrophic lateral sclerosis (ALS) (DeJesus-Hernandez et al. 2011, Renton et al. 2011).

Presently, there is no disease-modifying therapy for C9orf72 patients or other forms of FTD, ALS or combined ALS-FTD. Below we refer to patients carrying a C9orf72 mutation and suffering from ALS, FTD or combined ALS and FTD as “C9orf72 ALS/FTD” (the term “ALS-FTD” is used interchangeably). From current knowledge, gain of function toxicity of sense and antisense repeat RNA transcripts and/or non-conventional translation of both transcripts in all reading frames into five aggregating dipeptide repeat (DPR) proteins (poly-GA/-GP/-GR/-PA and -PR) (Mori, Arzberger et al. 2013, Mori, Weng et al. 2013) are main drivers of pathogenesis. The DPR proteins co-aggregate in abundant neuronal cytoplasmic inclusions. Poly-GA is the most abundant DPR species and forms amyloid-like twisted ribbons that likely are the core for co-aggregation of the other DPR proteins, which are all individually far more soluble (May et al. 2014, Guo et al. 2018). Although synergistic effects are likely driving disease pathogenesis, removing a single component may be sufficient to stop or at least delay disease progression.

Preventing the expression and translation of the (GGGGCC)ₙ repeat RNA using intrathecal injection of antisense oligonucleotides ameliorates behavioral phenotypes in mice (Jiang, J., et al. (2016). Neuron 90(3): 535-550.). While antisense oligonucleotides can specifically inhibit synthesis of new DPRs, they have no effect on the pre-existing DPR proteins, that start accumulating many years prior to disease onset (Vatsavayai, S. C., et al. (2016), Brain 139(Pt 12): 3202-3216.). The inventors hypothesize that removing DPR proteins and especially poly-GA by immunotherapy is beneficial in C9orf72 ALS/FTD, because DPRs are more toxic than the repeat RNA itself and clearing antibodies can potentially clear pre-existing aggregates.

Inclusions of poly-GA and other DPR proteins have been described by the inventors and others specifically in C9orf72 ALS/FTD cases. DPR inclusions precede disease onset and may contribute to the long prodromal stage with widespread smoldering brain atrophy in C9orf72 patients (Rohrer et al. 2015, Vatsavayai et al. 2016). Current research suggests that synergistic effects of different C9orf72-specific pathomechanisms eventually trigger the disease in a cascade-like manner (Edbauer and Haass 2016). Although poly-GA inclusions do not spatially correlate with neurodegeneration or TDP-43 pathology in C9orf72 patients, higher levels of poly-GA in the cerebellum is associated with FTD (Schludi et al. 2015). Upon individual expression, poly-GA forms twisted ribbons and sequesters large amounts of proteasomes that are stalled in an otherwise rare transition state (Guo et al. 2018). Among the different candidate mechanisms, only individual expression of poly-GA results in modest TDP-43 pathology in vitro (Khosravi et al. 2016, Nonaka et al. 2018) and in two mouse models (Zhang et al. 2016, Schludi et al. 2017), suggesting it is a main driver of neurodegeneration in vitro. Recent data from a primate model of TDP-43 pathology suggests that rodent caspases less efficiently cleave TDP-43 to generate aggregation-prone C-terminal fragments, which may explain the absence of large TDP-43 aggregates in C9orf72 mouse models and DPR expression experiments in primary rodent neurons (Yin et al., 2019).

Currently, several approaches are being pursued to develop a C9orf72-specific therapy. (i) Antisense oligonucleotides inhibit expression of DPR proteins and are beneficial in C9orf72 BAC transgenic mouse models (Jiang et al. 2016, Gendron et al. 2017). This would require regular life-long intrathecal injection of the antisense oligonucleotides in patients. A Phase 1 clinical trial is under way (NCT03626012). (ii) Interfering with G-quadruplex formation of the repeat RNA leads to modest decrease of DPR protein expression (Su et al. 2014, Simone et al. 2018). (iii) Several groups reported compounds that interfere with the non-conventional translation of the repeat RNA in absence of an ATG start codon, but the published compounds are likely too toxic for use in humans. A recent patent application disclosed additional more promising compounds such as metformin (WO2018/195110). (iv) The inventors of the present invention have shown that poly-GA antibodies block aggregation and spreading of poly-GA in cellular models (Zhou et al. 2017), although usability of such antibodies in in vivo models has not be demonstrated so far.

For neurodegenerative diseases, the poor delivery across the blood-brain barrier is rate-limiting. Although antibody engineering can increase antibody delivery from ˜0.05 to 0.5% (Yu et al 2017; Sci Transl Med 6: 261ra154), the antibody levels still need to be high. Low antibody trough levels and development of anti-drug antibodies are two factors known to limit the long-term efficacy of antibody therapy in oncology and inflammatory diseases (Kverneland et al 2018, Oncoimmunology 7: e1424674; Mazor et al. 2014, Aliment Pharmacol Ther 40: 620-628). Passive immunization typically results in 100-200 μg/ml serum levels that drop sharply within weeks in humans (Landen et al 2017; Alzheimers Dement (N Y) 3: 339-347; Sevigny et al 2016, Nature 537: 50-56). Maintaining antibody titers for a prolonged period might be achieved by active immunization, but this bears safety risks that for example unwanted T cell responses to active vaccination cause side effects, as observed. in a subset of AD patients immunized with A13 peptides that caused severe meningoencephalitis (Axelsen et al 2011, Vaccine 29: 3260-3269; Orgogozo et al 2003, Neurology 61: 46-54; Schenk et al 1999, Nature 400: 173-177). This suggests that a careful choice of the immunogen is essential.

### SUMMARY OF THE INVENTION

In a first aspect the present invention provides an immunogen for use in preventing or treating familial frontotemporal dementia (FTD), amyotrophic lateral sclerosis (ALS) and/or amyotrophic lateral sclerosis-frontotemporal dementia (ALS/FTD) in patients with C9orf72 repeat expansion comprising or consisting of a polypeptide consisting of dipeptide-repeats with a sequence selected from the group consisting of (Gly-Ala)ₐ, (Gly-Pro)ₐ, (Gly-Arg)ₐ, (Pro-Ala)ₐ and (Pro-Arg)ₐ, wherein a is an integer of 4 to 25, preferably 7 to 15, more preferably 8 to 12.

In a second aspect the present invention provides an immunogenic composition comprising or consisting of the immunogen of the first aspect of the invention, a pharmaceutically acceptable carrier and/or suitable excipient(s).

In a third aspect the present invention provides an immunogenic composition according to the second aspect of the invention for use in preventing or treating FTD and/or ALS.

In a fourth aspect the present invention provides a kit comprising the immunogen according to the first aspect of the invention, or the immunogenic composition according to the second aspect of the invention; and optionally at least one adjuvant. Optionally further comprising a container, and/or a data carrier, preferably comprising instructions for one or more of the first to third and fifth aspect of the present invention.

In a fifth aspect the present invention provides a method of treatment or prevention of FTD and/or ALS in a subject, wherein the method comprises administering an immunogen according to the first aspect of the invention or an immunogenic composition according to the second aspect of the invention.

In a sixth aspect the present invention provides a nucleic acid encoding the immunogen according to the first aspect of the invention.

In a seventh aspect the present invention provides a vector comprising the nucleic acid of the sixth aspect of the invention, preferably wherein the vector is a viral vector.

In an eight aspect the present invention provides an immunogen comprising or consisting of
a polypeptide comprising or consisting of dipeptide-repeats with a sequence selected from the group consisting of (Gly-Ala)ₐ, wherein a is an integer of 4 to 25, preferably 7 to 15, more preferably 8 to 12; and
a carrier protein, preferably tetanus toxoid (TT), HSP60, Concholepas concholepas hemocyanin (CCH), diphtheria toxin CRM197, diphtheria toxoid (DT), meningococcal outer membrane protein complex (OMPC), ovalbumin (OVA), keyhole limpet hemocyanin (KLH) or bovine serum albumin (BSA),
   wherein the carrier is non-covalently or covalently linked to the polypeptide, preferably by a spacer.

### LIST OF FIGURES

In the following, the content of the figures comprised in this specification is described. In this context, please also refer to the detailed description of the invention above and/or below.

FIG. 1 A) shows the immunization strategy and antibody response in wildtype (WT) and (GA)₁₄₉-CFP expressing transgenic mice (TG) as a model of C9orf72 ALS/FTD (Schludi et al. 2017). The age of the mice (in weeks) and the time points of immunization and blood sampling are indicated. The first immunization was applied at an age of 8 weeks followed by five booster injections up to an age of 28 weeks. Since poly-GA is expected to be poorly immunogenic we used either ovalbumin as an immunogenic carrier molecule (OVA-PEG-(GA)₁₀) that also keeps (GA)₁₀ soluble or a carrier-free self-aggregating (GA)₁₅ as immunogen. A 3-mer PEG spacer was used throughout FIGS. 1 to 5. B) shows the amount of anti-GA antibodies induced by the immunization of WT and TG mice with (GA)₁₅, (OVA)-PEG-(GA)₁₀ or PBS (control) as measured by ELISA using a GST-(GA)₁₅ antigen. Purified mouse anti-GA clone 1A12 was use as a reference standard to allow absolute quantification. A significant increase in anti-GA antibodies was observed in WT and TG-mice immunized with (OVA)-PEG-(GA)₁₀ but not with carrier-free (GA)₁₅. Thus, poly-GA without immunogenic carrier is not sufficient to induce a humoral immune response even after multiple boosting. The number of mice per group is indicated in the legend to the graph.

FIG. 2 shows the further characterization of the antisera from TG immunized mice with OVA-PEG-(GA)₁₀, (GA)₁₅ or PBS control according to the protocol in FIG. 1 (serum from week 29). A) shows immunoblots of HEK293 cells transfected with three poly-GA expressing construct and GFP control. (GA)₁₄₉-myc and (GA)₁₇₅-GFP express poly-GA from a synthetic gene using an AUG start codon (May et al., Acta Neuropathol 2014). (G₄C₂)₈₀ expresses poly-GA from the endogenous repeat sequence with 113 nucleotides upstream intronic sequence in the absence of an AUG start codon by RAN translation (Mori et al., EMBO Reports 2016). The serum (diluted 1:5000) from GA-CFP transgenic mice immunized with OVA-PEG-(GA)₁₀ shows poly-GA specificity similar to the anti-GA monoclonal antibody 1A12 (WO 2017/114660 A1). Calnexin is used as loading control (Enzo Life Sciences, ADI-SPA-860-F). B) Immunohistochemistry of sections from a C9orf72 patient and a healthy control in the molecular layer of the cerebellum. Monoclonal anti-GA (1A12) and antiserum (diluted 1:500) from OVA-PEG-(GA)₁₀ vaccinated mice detect neuronal cytoplasmic inclusions (some marked with arrows). (GA)₁₅ antiserum did not detect specific aggregates in the C9orf72 patient.

FIG. 3 shows the effect of the immunization of FIG. 1 on motor deficits in a transgenic mouse model overexpressing (GA)₁₄₉-CFP (Schludi et al., 2017, Acta Neuropathol. 2017 August; 134(2):241-254). Motor performance was assessed using weekly beam walk assay starting at 9 weeks of age before the onset of symptoms. The time required to cross the 58 cm long and 8 mm thick pole in the beam walk assay from a total of 4 runs from 2 consecutive weeks was averaged for the graph in A). In case a mouse dropped down, the time was recorded as 60 seconds. Only OVA-PEG-(GA)₁₀ immunization reduced the motor deficits in the transgenic mice (TG). WT mice needed a similar time to cross the pole regardless of treatment and age. PBS-treated TG mice needed significantly more time to complete the beam walk from week 15 onwards indicating motor deficits in those mice consistent with our previous report (Schludi et al., 2017, Acta Neuropathol. 2017 August; 134(2):241-254). The TG-mice immunized with (OVA)-PEG-(GA)₁₀ had an initial increase in beam walk time with a peak in week 17 (possibly due to lower antibody titer at this timepoint) and afterwards the performance improved almost back to levels of control mice (not significantly different from WT-PBS group). The TG-mice immunized with (GA)₁₅ overall performed as the PBS treated TG-mice. The effect of immunization on motor deficits (beam walk time) was in line with the degree of neuroinflammation and the amount of insoluble poly-GA aggregates measured in spinal cord (compare FIG. 4). Two-way ANOVA, Tukey's post hoc test was performed. The relevant comparisons of TG-PBS with WT-PBS, TG-OVA-PEG-(GA)₁₀ mice with TG-PBS and WT-PBS are depicted with * p<0.05, **, p<0.01, *** p<0.001, ns not significant. The number of mice per group is indicated in the legend to the graph. B) Average number of drop-downs per group from all aggregated runs. One-way ANOVA, Tukey's post hoc test **, p<0.01. Number of mice as in A).

FIG. 4 shows the effect of the vaccination on poly-GA aggregation and microglia activation in mice sacrificed after the behavioral analysis from FIG. 3. Post mortem analysis of the spinal cord of vaccinated GA-CFP and control mice. (A, B) Quantitative analysis of poly-GA aggregates in the spinal cord using immunohistochemistry (anti-GFP ThermoFisher A11122). (C) Immunoassay of poly-GA from the insoluble fraction as described in Schludi et al., Acta Neuropathol 2017. One-way ANOVA, Tukey's post hoc test *** p<0.001. Number of mice per group as in FIG. 1B. (D-F) Analysis of microglia activation using Iba1 immunohistochemistry (anti-Iba1, Wako 019-19741) in the spinal cord. (B, E and F) Image were analyzed from spinal cord sections at 1 mm intervals from n=3-4 mice per group. One-way ANOVA, Tukey's post hoc test * p<0.05, *** p<0.001.

FIG. 5 shows the effect of immunization strategy on A) body weight of immunized mice (number of mice per group as in FIG. 1) and B) leukocyte distribution in the spleen measured by flow cytometry (at the end of the study in week 32, n=5-7) suggesting that boosting regimen did not alter or impair the overall health and immune function of vaccinated mice. No significant differences were found using two-way ANOVA with Tukey's post hoc test.

FIG. 6 shows non-cell-autonomous effects of poly-GA on cytoplasmic TDP-43 mislocalization and aggregation. Fluorescently tagged poly-GA was expressed using a synthetic gene with an AUG start codon in the donor compartment of a co-culture model.

(A-C) Primary hippocampal neurons were transduced (after 4 days in to for another 4 days, DIV4+4) with GFP or (GA)₁₇₅-GFP, abbreviated as GA-GFP in the figure, and co-cultured with naïve primary neurons for 4 days. Endogenous TDP-43 and poly-GA aggregates in donor and receiver coverslips were analyzed by immunofluorescence (with C-terminal TDP-43 antibody from Cosmo Bio Co., Ltd., #TIP-TD-P09). (A) Schematic representation of co-culture experiments. (B) Cytoplasmic TDP-43 immunostaining is elevated not only in poly-GA transduced neurons, but also in the non-transduced receiver cells. Arrows and arrowheads indicate cells with cytoplasmic TDP-43 in GFP positive and negative cells, respectively. (C) Automated quantification of cells with cytoplasmic TDP-43 in GFP or (GA)₁₇₅-GFP transduced (donor), non-transduced (receiver) neurons using Columbus Acapella. Cells with and without GFP signal were analyzed separately (indicated by +/−). Two groups (GFP negative donor, GFP positive receiver) were excluded due to very high GFP transduction rat and very low GFP transmission rate. N=4 biological replicates. Note that (GA)175-GFP expression led to TDP-43 mislocalization even in neighboring cells without detectable GFP signal. Scatter plot with bar-graphs of mean±SD. One-way ANOVA with Tukey's multiple comparisons test. *** denotes p<0.001.

(D-F) A co-culture model in HeLa cells transfected with iRFP670 or (GA)₁₇₅-iRFP670 (abbreviated as iRFP and GA-iRFP in the figure) in the donor compartment and GFP-tagged TDP-43 lacking the nuclear localization signal (TDP-43_{ΔNLS}-GFP, K95A/K97A/R98A mutation as described by Winton et al., 2008) in donor and receiver compartment was used to promote TDP-43 aggregation. One day after separate transfection the extensively washed coverslips were co-cultured for an additional day. (D, E) Immunofluorescence staining and automatic quantification of TDP-43_{ΔNLS}-GFP aggregate number per cell in the donor and receiver and receiver compartment upon iRFP670 or (GA)₁₇₅-iRFP670 transfection using ImageJ. n=3 biological replicates. Scatter plot with bar-graphs of mean±SD. One-way ANOVA with Tukey's multiple comparisons test. ** denotes p<0.01, and *** denotes p<0.001. (F) Filter trap assay of SDS-insoluble TDP-43_{λNLS}-GFP aggregates in donor and receiver compartment upon iRFP670 or (GA)₁₇₅-iRFP670 transfection of donor cells. Scale bars denotes 20 μm.

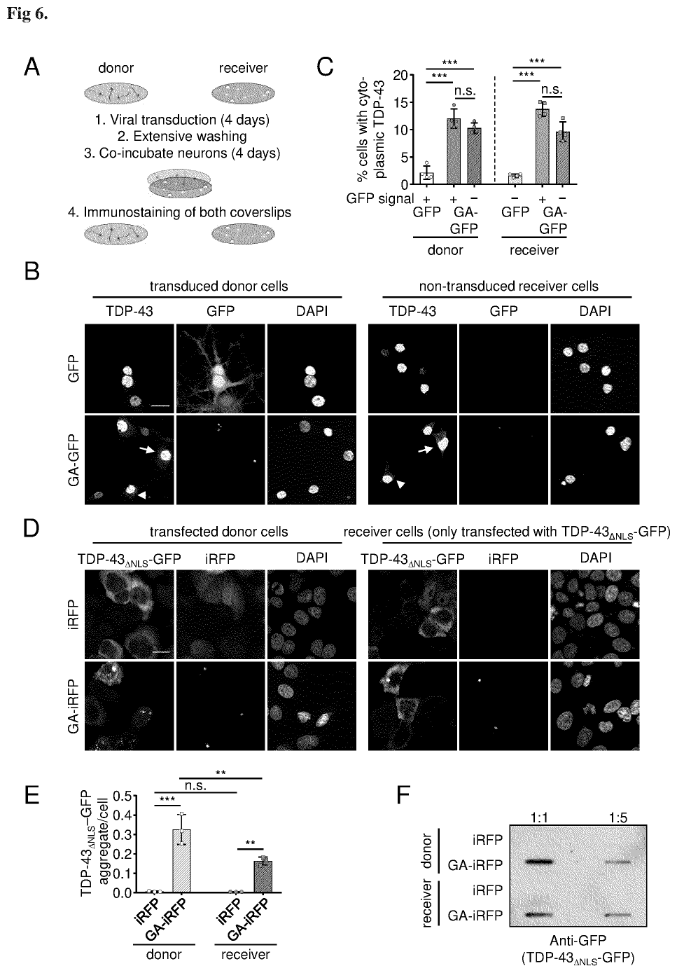

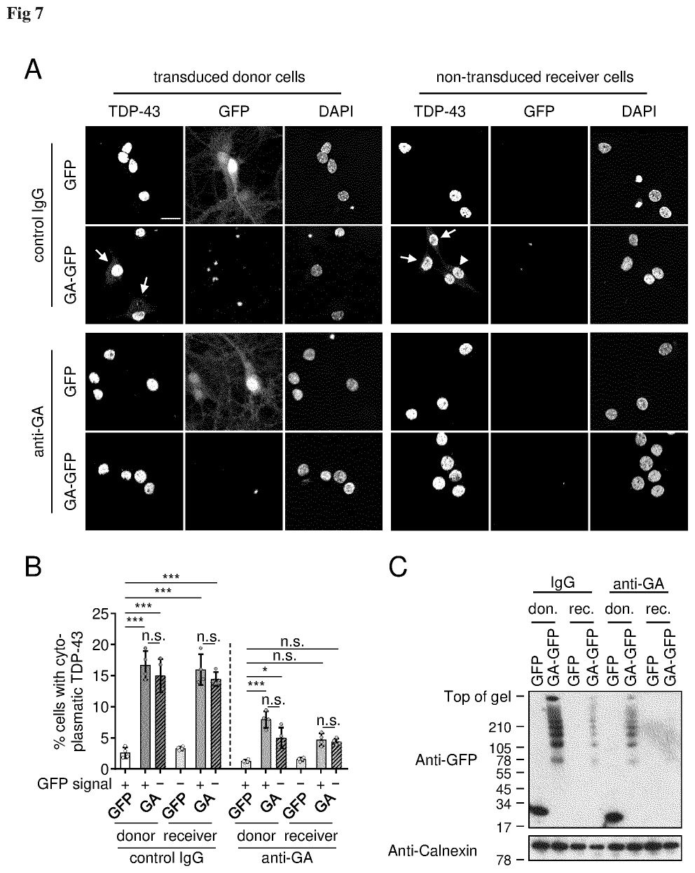

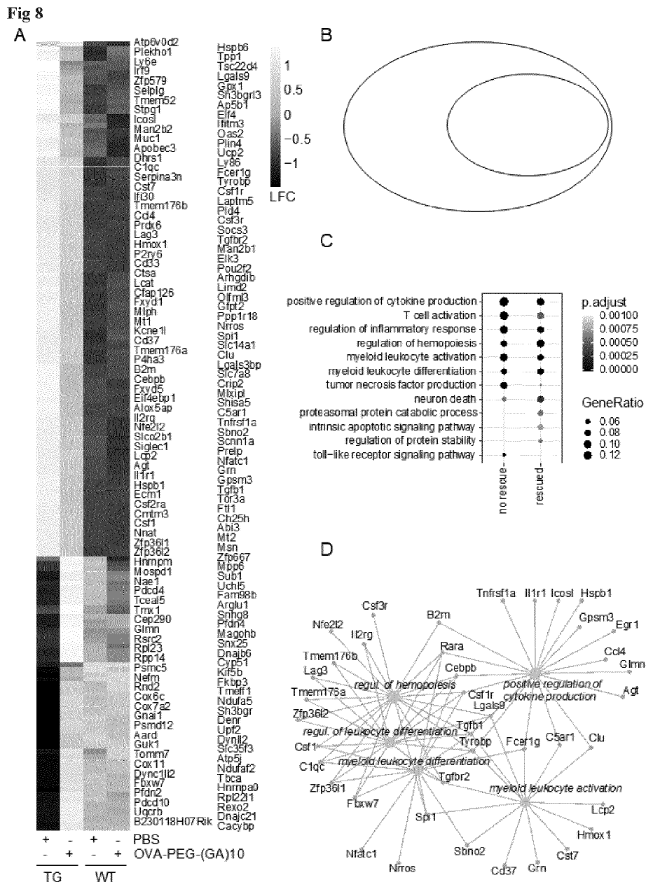

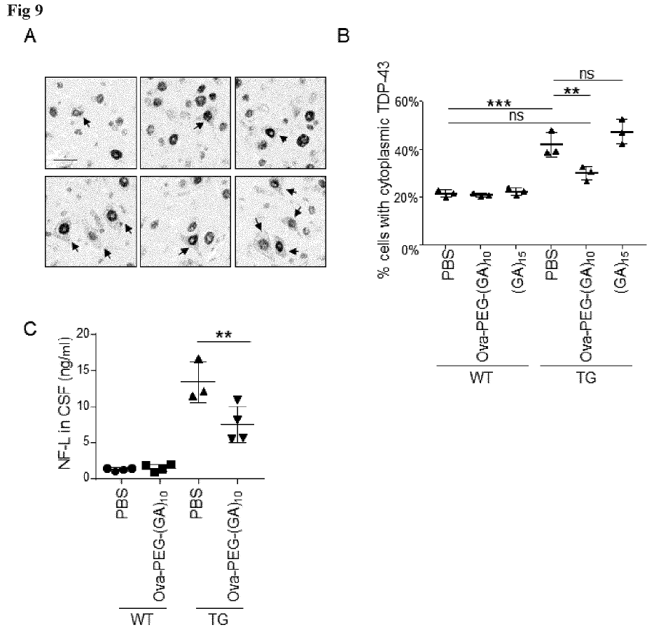

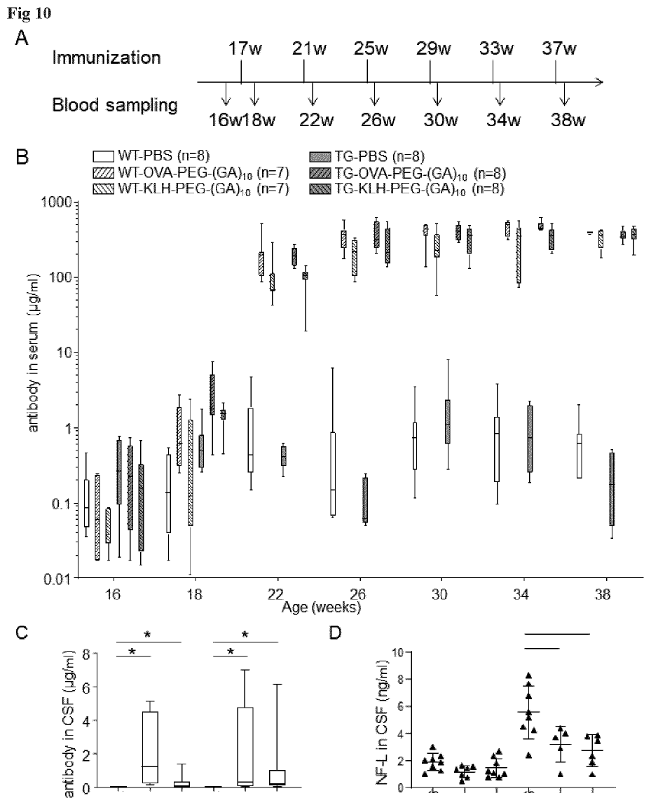

FIG. 7 shows that anti-GA antibodies block the non-cell-autonomous effects of poly-GA on TDP-43 mislocalization. Primary hippocampal neurons were transduced with GFP or (GA)₁₇₅-GFP (abbreviated as GA-GFP. DIV4+4 as in FIG. 6A) and then co-cultured for 7 days adding mouse IgG control or anti-GA antibody (mouse clone 5F2, Zhou et al., EMBO Mol Med 2017) at 1 μg/ml. (A) Confocal imaging revealed anti-GA antibody treatment reduces Poly-GA induced cytoplasmic mislocalization of endogenous TDP-43 in hippocampal neurons in both donor and receiver compartments. Arrows and arrowheads show cells with cytoplasmic TDP-43 in GFP positive and negative cells, respectively. Scale bar denotes 20 μm. (B) Automated quantification of cells with cytoplasmic TDP-43 in GFP or (GA)₁₇₅-GFP transduced cells. Cells with and without GFP signal were analyzed separately (indicated by +/−). As in FIG. 6C GFP-negative donor and GFP-positive receiver cells were excluded due to high transduction and low transmission rate of GFP. N=4 biological replicates. Scatter plot with bar-graphs of mean±SD. One-way ANOVA with Tukey's multiple comparisons test. * denotes p<0.05, and *** denotes p<0.001. (C) Immunoblotting shows reduced poly-GA levels upon anti-GA antibody treatment in both donor (don.) and receiver (rec.) compartment.

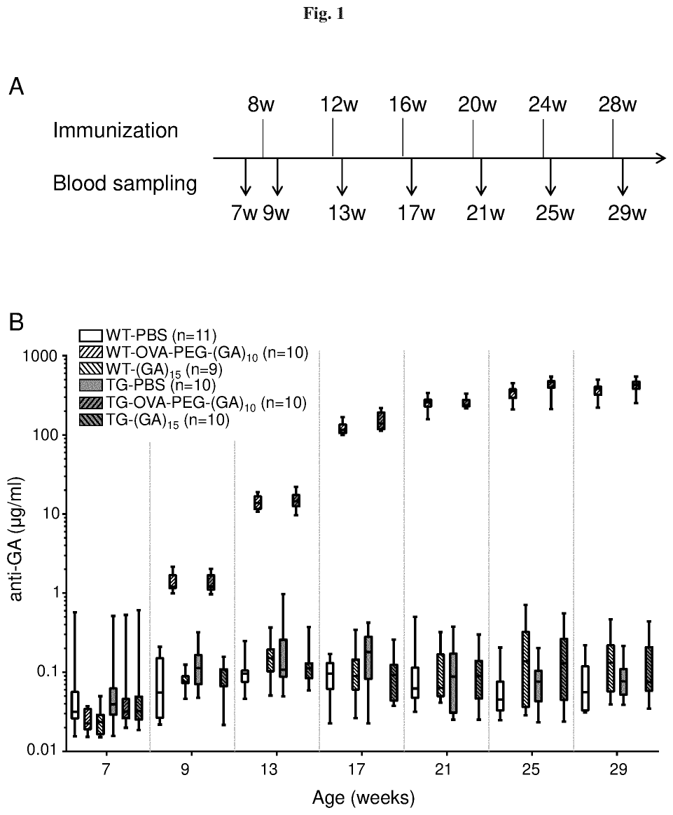

FIG. 8 shows transcriptome analysis of in wildtype (WT) and (GA)₁₄₉-CFP expressing transgenic mice (TG) immunized with OVA-PEG-(GA)₁₀ in comparison to PBS control as in FIG. 1. N=4 animals per group except for TG-PBS with N=3 animals. A) shows normalized expression levels of 164 genes that are differentially expressed genes in TG-PBS mice compared to WT-PBS controls and are significantly rescues by OVA-PEG-(GA)₁₀ immunization. Cut-off criteria are absolute log 2 fold-change >0.585 (i.e. 1.5 fold change) and p<0.05 after correction for multiple testing. The analysis was done using DESeq2 in R. B) shows a Venn diagram of expression changes comparing TG-PBS and WT-PBS groups and genes affected by OVA-PEG-(GA)₁₀ immunization in transgenic animals. C) shows a gene ontology analysis of differentially expressed genes in TG-PBS mice comparing genes significantly rescued by OVA-(GA)₁₀ immunization and non-rescued genes (absolute log 2 fold-change >0.585). The dot size and grey scale represent the fraction of the differentially expressed genes in each category and adjusted p-values, respectively. D) Network of the genes dysregulated in TG-PBS and significantly rescued in TG-OVA-(GA)₁₀.

FIG. 9 shows beneficial effects of OVA-PEG-(GA)₁₀ immunization on TDP-43 mislocalization and neuroaxonal damage of (GA)₁₄₉-CFP expressing transgenic mice (TG) and wildtype mice (WT) immunized as in FIG. 1A. A) shows representative immunofluorescence images of endogenous TDP-43 in the anterior horn of the spinal cord. In TG mice, more neurons show partial cytoplasmic mislocalization of TDP-43 (arrows), which is significantly higher compared to WT mice and is partially rescued by OVA-PEG-(GA)₁₀ immunization. Scale bar indicates 20 μm. B) shows quantification of cells with partial cytoplasmic mislocalization of TDP-43. Six images from spinal cord sections at 1 mm intervals were analyzed. N=3 mice per group. One-way ANOVA, Tukey's post hoc test. * p<0.05, ns not significant. F(_{5,12})=0.6533, p=0.6650; TG-PEG-Ova-(GA)₁₀ vs. TG-PBS p=0.0096; TG-(GA)₁₅ vs. TG-PBS p=0.4215. C) shows an immunoassay of neurofilament light chain (NFL) levels in cerebrospinal fluid, a clinically used marker of neuroaxonal damage. Samples were taken at week 25 after 5 immunizations of wildtype (WT) and (GA)₁₄₉-CFP expressing transgenic mice (TG) at 8, 12, 16, 20, 24 weeks of age analogous FIG. 1A. Animals were anesthetized by intraperitoneal administration of medetomidine (0.5 mg/kg), midazolam (5 mg/kg) and fentanyl (0.05 mg/kg). Cerebrospinal fluid (CSF) was collected from the cisterna magna according to the previously published methods (Schelle et al, Alzheimers Dement 2017). One-way ANOVA, Tukey's post hoc test, F(_{3,11})=1.911, p=0.1862. ** p<0.01. TG-Ova-(GA)₁₀ vs. TG-PBS p=0.0081.

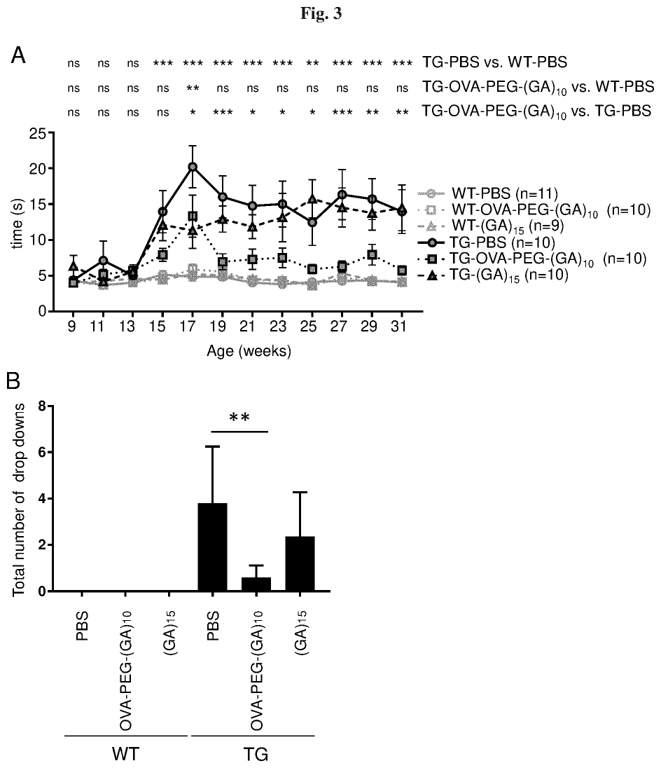

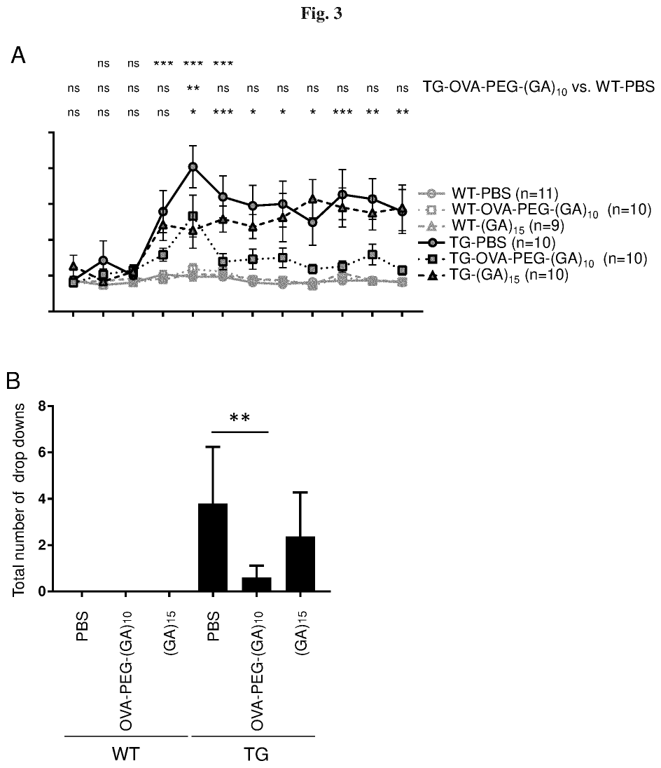

FIG. 10 shows the immunization strategy and antibody response in wildtype (WT) and symptomatic (GA)₁₄₉-CFP expressing transgenic mice (TG) using OVA-PEG-(GA)₁₀ or KT H-PEG-(GA)₁₀. A) The age of the mice (in weeks) and the time points of immunization and blood sampling are indicated. The first immunization was applied at 17 weeks-of-age (around the time when TG mice develop motor deficits, compare FIG. 3A), followed by five booster injections at week 21, 25, 29, 33 and 37. Serum was collected at weeks 16, 18, 22, 26, 30, 34 and 38. B) Anti-GA response measured by ELISA. Ovalbumin and KLH conjugates induce comparable anti-GA titers around ˜400 μg/ml similar to the cohort in FIG. 1. N as indicated in legend. In a mixed-effects model with Geisser-Greenhouse correction and Tukey's post-hoc test, the antibody response in TG-OVA-(GA)₁₀ vs. TG-KLH-(GA)₁₀ is not significantly different. C) Anti-GA antibodies are detected in the cerebrospinal fluid (collected at week 40 after 6 immunizations) in this cohort of OVA-PEG-(GA)₁₀ or KLH-PEG-(GA)₁₀ immunized wildtype (WT) and (GA)₁₄₉-CFP expressing transgenic mice. Kruskal-Wallis test, * p<0.01, TG-Ova-(GA)₁₀ vs. TG-PBS p=0.0174, TG-KLH-(GA)₁₀ vs. TG-PBS p=0.0236 D) shows an immunoassay of neurofilament light chain (NFL) levels in cerebrospinal fluid of this cohort of mice collected at week 40 after 6 immunizations. NFL is a clinically used marker of neuroaxonal damage. Immunization with KLH-conjugated (GA)₁₀ is at least as effective to suppress neuroaxonal damage as the OVA-conjugated antigen. N as in panel A). One-way ANOVA, Tukey's post hoc test, F(_{5,36})=2.818, p=0.0301. * p<0.05, *** p<0.001. TG-Ova-(GA)₁₀ vs. TG-PBS p=0.0111, TG-KLH-(GA)₁₀ vs. TG-PBS p=0.0008.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step.

As used in this specification and the appended claims, the singular forms “a”, “an”, and “the” include plural referents, unless the content clearly dictates otherwise.

As used herein, an “individual” means any mammal, reptile or bird that may benefit from the present invention. Preferably, an individual is selected from the group consisting of laboratory animals (e.g. mouse, rat or rabbit), domestic animals (including e.g. guinea pig, rabbit, horse, donkey, cow, sheep, goat, pig, chicken, duck, camel, cat, dog, turtle, tortoise, snake, or lizard), or primates including chimpanzees, bonobos, gorillas and human beings. It is particularly preferred that the “individual” is a human being.

A “patient” is any recipient of health care services. Typically, the patient is ill or injured, or susceptible to illness or injury or at risk of developing a disease (e.g. due to C9orf72 repeat expansion) and thus, in need of treatment by a physician, physician assistant, advanced practice registered nurse, veterinarian, or other health care provider. As used herein, a patient means any mammal, reptile or bird that may benefit from the invention described herein. Preferably, a “patient” is selected from the group consisting of laboratory animals (e.g. mouse or rat), domestic animals (including e.g. guinea pig, rabbit, horse, donkey, cow, sheep, goat, pig, chicken, camel, cat, dog, turtle, tortoise, snake, or lizard), or primates including chimpanzees, bonobos, gorillas and human beings. It is particularly preferred that the “patient” is a human being.

The term “tissue” as used herein, refers to an ensemble of cells of the same origin which fulfil a specific function concertedly. Examples of a tissue include but are not limited to nervous tissue, muscle tissue, bone, cartilage, connective tissue, and epithelial tissue. Multiple tissues together form an “organ” to carry out a specific function. Examples of an organ include but are not limited to brain, muscle, heart, blood, skeleton, joint, liver, kidney, stomach, and skin.

The term “cell” as used herein may either refer to a prokaryotic (e.g. a bacterial cell) or a eukaryotic cell (e.g. a fungal, plant or animal cell). Multicellular organisms comprise several types of cells differentiated to fulfil different function in said organism. These include but are not limited to stem cell, embryonic stem cells, cells of the nervous system, blood cells, cells of the immune system, mesenchymal cells, epithelial cells, interstitial cells, metabolism and storage cells, gland cells, extracellular matrix cells, contractile cells, pigment cells, germ cells and tumor cells. The term “cell” as used herein also refers to those cells being removed from their natural environment, such as isolated primary cell or cell lines of any of the above named cell types. Typically, cells such as bacterial cell, yeast cell, isolated primary cell or cell line are used in biotechnological assays. In the context of the present invention isolated primary cell or cell lines are preferably of mammalian origin.

The terms “polynucleotide” and “nucleic acid” are used interchangeably herein. Nucleic acid molecules are understood as a polymeric or oligomeric macromolecule made from nucleotide monomers. Nucleotide monomers are composed of a nucleobase, a five-carbon sugar (such as but not limited to ribose or 2′-deoxyribose), and one to three phosphate groups. Typically, a polynucleotide is formed through phosphodiester bonds between the individual nucleotide monomers. In the context of the present invention referred to nucleic acid molecules include but are not limited to ribonucleic acid (RNA), deoxyribonucleic acid (DNA), and mixtures thereof such as e.g. RNA-DNA hybrids, as well as cDNA, genomic DNA, recombinant DNA, cRNA and mRNA. A nucleic acid may consist of an entire gene, or a portion thereof, the nucleic acid may also be a microRNA (miRNA) or small interfering RNA (siRNA). MiRNAs are short ribonucleic acid (RNA) molecules, on average only 22 nucleotides long, found in all eukaryotic cells.

The term “CpG oligodeoxynucleotides” (CpG ODN) as used herein refers to short single-stranded DNA molecules that contain a cytosine triphosphate deoxynucleotide followed by a guanine triphosphate deoxynucleotide. CpG ODNs with unmethylated CpG motifs act as immunostimulants. CpG motifs, in particular unmethylated CpGs, are pathogen-associated molecular patterns due to their abundance in microbial genomes but their rarity in vertebrate genomes. The CpG motif is recognized by pattern recognition receptors such as Toll-Like Receptor 9 (TLR9), which is constitutively expressed only in B cells and plasmacytoid dendritic cells (pDCs) in humans and other higher primates thereby acting as immunostimulant. Synthetic CpG ODNs may differ from microbial DNA in that they have a partially or completely phosphorothioated backbone instead of the typical phosphodiester backbone and a poly G tail at the 3′ end, 5′ end, or both. The backbone modification results in increased resistance to nucleases, whereas the poly-G tail enhances cellular uptake of the CpG ODNs. Numerous CpGs are known in the art as immunostimulants. The CpG ODNs used to exercise the present invention are preferably immunostimulating CpG ODNs usable as adjuvants. Preferably the CpG ODNs are synthetic CpG ODNs with a nuclease resistant backbone and/or poly G tail at the 3′ and/or 5′ end. Preferred CpG ODNs are ODN 2006, ODN 1668, ODN 2007, ODN 2216, ODN D35 and ODN K3, more preferably ODN 2006 and ODN 1668 (Hartmann, G., et al. (2000); J Immunol 164(3): 1617-1624 and Heit, A., et al. (2004), J Immunol 172(3): 1501-1507.).

The term “open reading frame” (ORF) refers to a sequence of nucleotides, that can be translated into amino acids. Typically, such an ORF contains a start codon, a subsequent region usually having a length which is a multiple of 3 nucleotides, but does not contain a stop codon (TAG, TAA, TGA, UAG, UAA, or UGA) in the given reading frame. Typically, ORFs occur naturally or are constructed artificially, i.e. by gene-technological means. An ORF codes for a peptide, polypeptide, or protein where the amino acids into which it can be translated forms a peptide-linked chain.

The terms “gene expression” or “expression” are used interchangeably herein and refer to the process by which the genetic information is used to synthesize a functional gene product. Typically, such gene product is a peptide, polypeptide, or protein, or a nucleic acid such as a ribosomal RNA (rRNA), transfer RNA (tRNA) or small nuclear RNA (snRNA). Gene expression includes the steps of transcription, RNA splicing, translation, and post-translational modification. Preferably, the term is used to refer to the synthesis of a peptide, polypeptide or protein. Thus, the term “detection of expression” is preferably used to refer to the detection of expression of a peptide, polypeptide or protein. Such detection can be carried out by art known methods, in particular by using ligands specifically binding to the peptide, polypeptide or protein.

Typically in protein synthesis, a DNA sequence encoding a gene is first transcribed into mRNA from which the introns are removed via RNA splicing and the exons are joined and subsequently translated to produce an amino acid chain, which then folds into a protein.

The term “transcription” refers to the process wherein a particular segment of DNA, typically a gene, is transcribed into RNA by the enzyme RNA polymerase. During transcription, a DNA sequence is read by an RNA polymerase, which produces a complementary, antiparallel RNA strand. As opposed to DNA replication, transcription results in an RNA complement that includes uracil (U) in all instances where thymine (T) would have occurred in a DNA complement. If the gene transcribed encodes a protein, the result of transcription is a pre-messenger RNA (mRNA) or mRNA molecule, which will then be translated into a peptide, polypeptide or protein. Alternatively, the transcribed gene may encode for either non-coding RNA genes (such as microRNA, lincRNA, etc.) or ribosomal RNA (rRNA) or transfer RNA (tRNA), other components of the protein-assembly process, or other ribozymes. “RNA splicing” occurs concurrently or after the transcription process and refers to the process wherein the “introns” comprised in the pre-mRNA are removed and the “exons” are covalently joined.

The term “intron” refers to any nucleotide sequence within a gene which is removed by RNA splicing. In the art, the term intron is typically used to refer to both, the DNA sequence within a gene and the corresponding sequence in the RNA transcript which is removed by RNA splicing.

Sequences that are joined together in the final mature RNA after RNA splicing are referred to as “exons”. Again, the term exon is typically used in the art to refer to both, the DNA sequences within a gene and the corresponding sequences in the RNA transcript which are joined during RNA splicing after removal of the intron.

Ribosomes facilitate the process of “translation” of mRNA into an amino acid chain by inducing the binding of tRNAs with complementary anticodon sequences to that of the mRNA. The tRNAs carry specific amino acids that are chained together into a polypeptide as the mRNA passes through and is “read” by the ribosome. Typically, translation is an AUG-dependent process, wherein an AUG codon of the mRNA (corresponding to an ATG codon of the DNA) is recognized as translation initiation site resulting in methionine being the first amino acid in the produced amino acid chain. However, also AUG-independent translation mechanism exist, e.g. wherein the methionine tRNA interacts with a codon complementary to only two nucleotides. In viruses AUG-independent translations mechanism includes the use of internal ribosome entry sites (IRES) which structurally mimic the initiator tRNA and manipulate the ribosomes to initiate the translation at a non-AUG site.

The term “RAN-translation” (Repeat-associated non-AUG translation) refers specifically to an AUG-independent translation mechanism of nucleotide repeats. In a strict sense translation is believed to be initiated directly within the repeat, but for several repeat expansion disorders low-efficiency initiation from near-AUG codons (e.g. CUG) upstream of the repeat region has been reported (e.g. Tabet et al., Nat Commun. 2018 Jan. 11; 9(1):152; Kearse et al, 2016, Mol Cell. 2016 Apr. 21; 62(2):314-322), which may be promoted by the repeat expansion. In the prior art it has been shown that RAN-translation occurs for exonic RNA comprising, trinucleotide, tetranucleotide and pentanucleotide repeats. In the work leading to the present invention, it was surprisingly shown that also intronic RNA transcribed from hexanucleotide repeats, present in the genome could also be translated by RAN-translation. Polypeptides which were RAN-translated of such nucleotide repeats may (but do not necessarily have to) differ from peptides or polypeptides that were translated in an AUG-dependent manner in that they lack the initial methionine. For instance, RAN-translation may be facilitated in that the presence of said nucleotide repeats promotes the formation of a hairpin structure which subsequently triggers the RAN-translation initiated within or near the repeat.

The terms “amino acid chain” and “polypeptide chain” are used synonymously in the context of the present invention.

In the context of the present invention, the term “peptide” refers to a short polymer of amino acids linked by peptide bonds. It has the same chemical (peptide) bonds as proteins, but is commonly shorter in length. The shortest peptide is a “dipeptide”, consisting of two amino acids joined by a single peptide bond. There can also be a tripeptide, tetrapeptide, pentapeptide, etc. Peptide may also have a length of up to 8, 10, 12, 15, 18 or 19 amino acids. A peptide has an amino end and a carboxyl end, unless it is a cyclic peptide.

In the context of the present invention, the term “dipeptide repeat (DPR)” refers to a dipeptide of two amino acids joined by a single peptide bond which is duplicated several times to form a longer peptide or a polypeptide, i.e. a dipeptide repeat may comprise 2, 3, 4, 5, 6, 7, 8, 9, 10, 11, 12, 13, 14, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000 or more dipeptides joined together by peptide bonds. Preferably, the dipeptide repeats used for immunization in the present invention are in the range of 2 to 30, 3 to 29, 3 to 28, 4 to 27, 4 to 26, 4 to 25, 5 to 24, 5 to 23, 5 to 22, 6 to 21, 6 to 20, 6 to 19, 7 to 18, 7 to 17, 7 to 16, 7 to 15, 8 to 14, 8 to 13, 8 to 12, 8 to 11 and 8 to 10, preferably 4 to 25, more preferably 7 to 15, even more preferably 8 to 12. For immunization DPR antigens are synthesized chemically or using a DNA vector for DNA vaccination. The very long “dipeptide repeats” found in C9orf72 mutation carriers are the translation products of hexanucleotide repeats. The meaning of this term is further described below. It requires inter alia that the smallest repeat unit has a length of six nucleotides, e.g. in the nucleotide sequence “CGCGCGCGCGCG” (SEQ ID NO: 001) the smallest repeat unit is “CG” and thus this sequence is a dinucleotide repeat. On the other hand in the nucleotide sequence GGGCCCGGGCCC (SEQ ID N: 002) the smallest repeat unit is “GGGCCC” and thus this is a hexanucleotide repeat. A hexanucleotide that does not comprise a STOP codon will thus encode a dipeptide and a string of repeats of such hexanucleotides will encode a dipeptide repeat. Due to the degeneration of the genetic code it is possible that a nucleotide sequence that fulfills the criterions of being a hexanucleotide repeat encodes a dipeptide of identical amino acids, e.g. the smallest repeat unit of the nucleotide sequence “GGTGGCGGTGGC” (SEQ ID NO: 003) is “GGTGGC”, which encodes Gly-Gly. However, it is preferred that the dipeptide comprises two different amino acids, e.g. (Gly-Ala), (Gly-Pro), (Gly-Arg), (Ala-Pro), (Pro-Arg), Gly-Leu), (Ala-Trp), (Pro-Gly), (Ala-Gln), preferably (Gly-Ala). Otherwise, i.e. if the amino acids of a dipeptide could be identical, it could not be determined for a six amino acid long peptide that there are three dipeptide repeats but such sequence would be considered by the skilled person to be either a hexapeptide or a sixfold repeat of a monomer. Accordingly, the term “dipeptide repeat (DPR)” refers to a dipeptide of two different amino acids joined by a single peptide bond which is duplicated several times to form a longer peptide or a polypeptide, i.e. a dipeptide repeat may comprise 2, 3, 4, 5, 6, 7, 8, 9, 10, 11, 12, 13, 14, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000 or more dipeptides joined together by peptide bonds. The N-terminal part of DPR proteins resulting from potential near-AUG initiation upstream of the (GGGGCC)ₙ repeat has not been fully resolved. Translation of the full length repeat sequence can additionally result in short C-terminal peptide extensions (Mori et al., Acta Neuropathol. 2013 December; 126(6):881-93; and Zu et al., PNAS Dec. 17, 2013 110 (51) E4968-E4977). In addition, frame-shifting may occur during repeat translation (Tabet et al., Nat Commun. 2018 Jan. 11; 9(1):152).

The term “polypeptide” refers to a single linear chain of amino acids bonded together by peptide bonds and preferably comprises at least about 20 amino acids. A polypeptide can be one chain of a protein that is composed of more than one chain, or it can be the protein itself if the protein is composed of a single chain.

The term “protein” refers to a molecule comprising one or more polypeptides that resume a secondary and tertiary structure and additionally refers to a protein that is made up of several polypeptides, i.e. several subunits, forming quaternary structures. The protein has sometimes non-peptide groups attached, which can be called prosthetic groups or cofactors.

An “isolated peptide”, “isolated polypeptide”, or “isolated protein” refers to a peptide, polypeptide or protein which has been removed from its natural environment in a cell such that other cellular material normally nearby is not present anymore. In the context of the present invention, also peptides, polypeptides or proteins produced outside their natural cellular environment, e.g. via chemical means or via recombinant means in a non-natural environment, are considered as isolated peptides, polypeptides or proteins.

Polypeptides or proteins (including protein derivatives, protein variants, protein fragments, protein segments, protein epitopes and protein domains) can be further modified by chemical modification. Hence, a chemically modified polypeptide may comprise chemical groups other than the residues found in the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g. one or more of enhanced stability, increased biological half-life, or increased water solubility. Chemical modifications include without limitation: PEGylation, glycosylation of non-glycosylated parent polypeptides. Such chemical modifications applicable to the variants usable in the present invention may occur co- or post-translational.

An “antigenic protein” as referred to in the present application is a polypeptide as defined above which contains at least one epitope. An “antigenic fragment” of an antigenic protein is a partial sequence of said antigenic protein comprising at least one epitope. For immunization purposes only those parts of a protein are relevant which elicit an immune response. Therefore, the nucleic acid construct does not need to encode the full-length antigenic protein as it is found in e.g. a diseased cell, a cancer cell, or a pathogen. A shortened fragment of such a protein is sufficient as long as its amino acid sequence comprises the epitope or epitopes responsible for the recognition of the antigenic protein by the immune system. The term “antigen” as used herein refers to any molecule or part of a molecule, including but not limited nucleic acid, amino acid, peptide, polypeptide, protein, carbohydrate, and lipid, to which a ligand of the invention binds.

The term “epitope” as used herein refers to an antigenic determinant, which is part of an antigen that is specifically bound by a ligand of the invention, preferably an antibody or antigen binding-fragment thereof. Epitopes typically consist of chemically active surface groupings of molecules such as amino acids or sugar side chains and usually have specific three-dimensional structural characteristics, as well as specific charge characteristics. The epitopes of an antigen may be a conformational epitope or a non-conformational, i.e. a linear, epitope. Conformational and non-conformational epitopes are distinguished in that the binding to the former but not the latter is lost in the presence of denaturing solvents. A conformational epitope is composed of discontinuous sections of the antigen's amino acid sequence. These epitopes interact with the ligand based on the 3D surface features and shape or tertiary structure of the antigen. Most epitopes are conformational. By contrast, linear epitopes interact with the ligand based on their primary structure. A linear epitope is formed by a continuous sequence of amino acids from the antigen. Conformational epitopes preferably comprise between 8 and 20 discontinuous amino acids, preferably between 8 and 15 amino acids. Linear epitopes have a length of between 6 to 20 amino acids, more preferably between 8 and 15 amino acids.

Peptides, polypeptides or proteins may be detected via various methods including but not limited to a filter trap assay, Western Blotting, enzyme-linked immunosorbent assay (ELISA), Immunohistochemistry (IHC), Immunocytochemistry (ICC), and size exclusion chromatography (SEC).

“Immunostaining” including but not limited to immunohistochemistry (IHC) or immunocytochemistry (ICC), is an antibody-based method to detect a specific protein in a sample. The term immunostaining was originally used to refer to the immunohistochemical staining of tissue sections. Now however, immunostaining encompasses a broad range of techniques used in histology, cell biology, and molecular biology that utilize antibody-based staining methods. While the first cases of IHC staining used fluorescent dyes, other non-fluorescent methods using enzymes such as peroxidase and alkaline phosphatase are now used more often. These enzymes are capable of catalyzing reactions that give a colored product that is easily detectable by light microscopy. Alternatively, radioactive elements can be used as labels, and the immunoreactions can be visualized by autoradiography. Tissue preparation or fixation is essential for the preservation of cell morphology and tissue architecture. Inappropriate or prolonged fixation may significantly diminish the antibody binding capability. Many antigens can be successfully demonstrated in formalin-fixed paraffin-embedded tissue sections. Optimization of fixation methods and times, pre-treatment with blocking agents, incubating antibodies with high salt, and optimizing post-antibody wash buffers and wash times may be important for obtaining high quality immunostaining.

“Western blotting” or “immunoblotting”, which are used interchangeably herein, allows the detection of specific proteins (native or denatured) from extracts made from cells or tissues, before or after any purification steps. Proteins are generally separated by size using gel electrophoresis before being transferred to a synthetic membrane (typically nitrocellulose or PVDF) via dry, semi-dry, or wet blotting methods. The membrane can then be probed using antibodies using methods similar to immunohistochemistry, but without a need for fixation. Detection is typically performed using peroxidase linked antibodies to catalyze a chemiluminescent reaction. Western blotting is a routine molecular biology method that can be used to semi quantitatively or quantitatively compare protein levels between extracts. The size separation prior to blotting allows the protein molecular weight to be gauged as compared with known molecular weight markers. Western blotting is an analytical technique used to detect specific proteins in a given sample of tissue homogenate or extract. It uses gel electrophoresis to separate proteins by the length of the polypeptide (denaturing conditions) or by the 3-D structure of the protein (native/non-denaturing conditions).

The “enzyme-linked immunosorbent assay (ELISA)” is a diagnostic method for quantitatively or semi-quantitatively determining protein concentrations from blood plasma, serum or cell/tissue extracts in a multi-well plate format (usually 96-wells per plate). Broadly, proteins in solution are adsorbed to ELISA plates. Antibodies specifically binding to the protein of interest are used to probe the plate.

“Electron microscopy (EM)” can be used to study the detailed micro architecture of tissues or cells. “Immuno-EM” allows the detection of specific proteins in ultrathin tissue sections.

Antibodies labeled with heavy metal particles (e.g. gold) can be directly visualized using transmission electron microscopy.

A “marker”, “tag”, or “label” is any kind of substance which is able to indicate the presence of another substance or complex of substances. The marker can be a substance that is linked to or introduced in the substance to be detected. Detectable markers are used in molecular biology and biotechnology to detect e.g. a protein, a product of an enzymatic reaction, a second messenger, DNA, interactions of molecules etc. Examples of suitable marker or labels include a fluorophore, a chromophore, a radiolabel, a metal colloid, an enzyme, or a chemiluminescent or bioluminescent molecule. Examples of fluorophores include various forms of green fluorescent protein (GFP) such as EnGFP, RFP, CYP, BFP, YFP, dsRed etc., phycobiliproteins (allophycocyanin, phycocyanin, phycoerythrin and phycoerythrocyanin), fluorescein (fluorescein isothiocyanate, FITC), rhodamine (tetramethyl rhodamine isothiocyanate, TRITC), and cyanine dyes (such as C2, Cy3 Cy5, Cy7). Examples of radiolabels include ³H, ¹⁴C, ³²P, ³³P, ³⁵S, ^{99m}Tc or ¹²⁵I. Examples of enzymes include luciferase, beta-galactosidase, horseradish peroxidase, alkaline phosphatase, glucose oxidase, and urease.

Different types of chemical labels or tags can be conjugated to secondary or primary antibodies and other molecules to facilitate their visualization (i.e., detection and measurement) by various methods. Radioisotopes were used extensively in the past, but they are expensive, have a short shelf-life, offer no improvement in signal to noise ratio and require special handling and disposal. Enzymes and fluorophores have largely replaced radioactive isotopes as detectable tags for assays. A number of advancements in reagents and instrumentation make these newer technologies more versatile and powerful. Enzymatic tags such as horseradish peroxidase (HRP) are most commonly used for blotting, immunoassays and immunohistochemistry methods. Fluorescent tags are used predominately for cellular imaging, nucleic acid amplification and sequencing and microarrays; however, fluorescence technology is developing rapidly for application in all types of assays.

The term “expression level” refers to the amount of gene product (e.g. DPR) present in the body or a sample at a certain point of time. The expression level can e.g. be measured/quantified/detected by means of the amounts of the protein or of the mRNA encoding the protein. For example the expression level can be quantified by normalizing the amount of gene product of interest (e.g. DPR) present in a sample with the total amount of gene product of the same category (total protein or mRNA) in the same sample or in a reference sample (e.g. a sample taken at the same time from the same individual or a part of identical size (weight, volume) of the same sample) or by identifying the amount of gene product of interest per defined sample size (weight, volume, etc.). The expression level can be measured/quantified/detected by means of any method as known in the art, e.g. methods for the direct detection and quantification of the gene product of interest (such as mass spectrometry) or methods for the indirect detection and measurement of the gene product of interest that usually work via binding of the gene product of interest with one or more different molecules or detection means (e.g. primer(s), probes, antibodies, scaffold-proteins) specific for the gene product of interest (e.g. DPR). Preferably, the expression level is determined on the basis of the protein rather than on the basis of the mRNA.

The term “toxicity” as used herein refers to the degree to which a compound/substance can damage an organism or a substructure of the organism, such as a cell (cytotoxicity), tissue or an organ. Accordingly, the term “toxic effect” refers to the damaging effect a compound/substance has on an organism, organ, tissue or cell. A compound may exhibit a toxic effect in that it damages the function and/or structure of an organism, organ, tissue or cell, which may result in an altered function or a loss of function of certain elements or parts of the organism, organ, tissue or cell, or may even result in the death of said organism, organ, tissue or cell. The term “toxic compound” thus, refers to a substance, e.g. a nucleic acid, a peptide (e.g. DPR), polypeptide or protein, or a chemical substance or compound, which exhibits a toxic effect on the organism, organ, tissue or cell.

The toxicity or the toxic effect of a compound may be measured using one of various viability assays known in the art including but not limited to formazan-based assays (MTT/XTT), Lactate dehydrogenase (LDH) assay, ATP test, Calcein AM, Clonogenic assay, Ethidium homodimer assay, evans blue, Fluorescein diacetate hydrolysis/Propidium iodide staining (FDA/PI staining), Flow cytometry, TUNEL assay, with green fluorescent protein (GFP), methyl violet, propidium iodide, trypan blue, or resazurin. Further, DNA stainings may be used to differentiate between necrotic, apoptotic and normal cells.

The term “disease” and “disorder” are used interchangeably herein, referring to an abnormal condition, especially an abnormal medical condition such as an illness or injury, wherein a cell, a tissue, an organ, or an individual is not able to efficiently fulfil its function anymore. Typically, but not necessarily, a disease is associated with specific symptoms or signs indicating the presence of such disease. The presence of such symptoms or signs may thus, be indicative for a cell, a tissue, an organ, or an individual suffering from a disease. An alteration of these symptoms or signs may be indicative for the progression of such a disease. A progression of a disease is typically characterized by an increase or decrease of such symptoms or signs which may indicate a “worsening” or “bettering” of the disease. The “worsening” of a disease is characterized by a decreasing ability of a cell, tissue, organ or individual/patient to fulfil its function efficiently, whereas the “bettering” of a disease is typically characterized by an increase in the ability of a cell, tissue, an organ or an individual/patient to fulfil its function efficiently. A cell, a tissue, an organ or an individual being “susceptible” to a disease is in a healthy state but especially vulnerable to the emergence of a disease, e.g. due to genetic predisposition, lacking vaccination, poorly developed or immature immunity, poor nutritional status, or the like. The outbreak of the disease may still be prevented by prophylaxis or pre-cautionary treatment. A cell, a tissue, an organ or an individual may be “suspected of having” a disease wherein said cell, tissue, organ or individual typically shows early or weak signs or symptoms of such disease. In such case, the onset of the disease may still be prevented or its progression may be reduced or prevented by treatment.

As used herein, “detect”, “detecting”, or “detection” of a disease or disorder refers to establishing the presence or absence of a disease in a patient. For instance, a moiety used in the detection of a disease is able to identify the presence or absence of an indicator of a disease in a sample or in an individual or patient. For instance, a disease may be detected by means of a ligand or a tagged ligand interacting with, i.e. binding to or forming a complex with, a disease specific nucleic acid or peptide, polypeptide or protein. A disease may also be detected by means of an inhibitor blocking the mechanism of action underlying the disease and thus, altering the symptoms such that the underlying disease may be identified.

As used herein, “treat”, “treating”, “treatment” or “therapy” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in an individual that has previously had the disorder(s); and (e) limiting or preventing recurrence of symptoms in individuals that were previously symptomatic for the disorder(s). Accordingly, a therapy treats a disease or disorder, or the symptoms of a disease or disorder by accomplishing one or more of above named effects (a)-(e). For instance, a disease may be treated by means of inhibiting/blocking the mechanism of action underlying the disease, e.g. via compounds that inhibit the expression, aggregation or toxicity (such as inflammation) of disease specific polypeptide/proteins (e.g. DPR) or by inhibiting further processes said polypeptide/protein is involved in. A disease may also be treated by activating the patients' immune system, e.g. via active immunization, or by supporting the patients' immune system, e.g. via passive immunization.

As used herein, “prevent”, “preventing”, “prevention”, or “prophylaxis” of a disease or disorder means preventing that such disease or disorder occurs in a patient. Accordingly, a compound having a prophylactic effect prevents the onset of a disease or disorder in a patient. For instance, a disease or disorder may be prevented by immunizing, e.g. actively or passively immunizing, a healthy individual such that the onset of disease is avoided or delayed. This includes ameliorating disease symptoms due to prophylactic intervention. Alternatively, a disease or disorder may be prevented through ligands or inhibitors undermining the function of disease related molecules or processes, e.g. via ligands or inhibitors that block the expression or aggregation of disease specific polypeptide/proteins (e.g. DPR) or by inhibiting further processes said polypeptide/protein is involved in.

The term “immunization” refers to the process of activating, strengthening or boosting the immune system of an individual against an agent (“vaccine”) which typically causes or induces a disease or disorder. Hence, by immunizing a healthy individual against said agent, the onset of a disease or disorder may be prevented. Immunizing a patient suffering from a disease or disorder may treat said disease or disorder or may prevent the further progression of said disease. Immunization may be achieved through various techniques, most commonly immunization is achieved through vaccination of the healthy individual or the patient suffering from a disease or disorder.

The term “passive immunization” refers to the process wherein pre-synthesized elements of the immune system are transferred to an individual/patient such that the body does not need to produce these elements itself. Passive immunization aims at the treatment of a disease or the prevention of the progression of a disease, in particular in cases where the patient is not able to combat such disease or disorder due to an inefficient immune system (e.g. deficient immune system, or unrecognizable immunogen, e.g. of a tumor). For instance, antibodies (e.g. animal or humanized antibodies produced in vitro by cell culture) directed against a disease-specific immunogen, or nucleic acids encoding said antibody and allowing for its expression, are means of passive immunization. The term “active immunization” refers to the immunization via the introduction of a foreign molecule (immunogen) into the body, which causes the body itself to generate an immune response against the immunogen. Active immunization aims at the prophylaxis of a disease or the prevention of the progression of a disease, as the immune system of an individual is primed/activated/strengthened to react against said immunogen resulting in a more efficient or faster immune response to the immunogen. The principle underlying active immunization is the generation of an immunological “memory”. In the context of neurodegenerative diseases where the cellular antigen (e.g. DPR) is continuously present, the aim of active immunization is to maintain high antibody levels continuously by life-long, regular immunization. Challenging an individual's immune system with e.g. a vaccine comprising a disease specific immunogen, induces the formation and/or propagation of immune cells which specifically recognize the immunogen comprised by the vaccine. At least a part of said immune cells remains viable for a period of time which can extend to 10, 20 or 30 years after vaccination. If the individual's immune system encounters the immunogen again within the aforementioned period of time, the immune cells generated by vaccination are reactivated and enhance the immune response against the immunogen as compared to the immune response of an individual which has not been challenged with the vaccine and encounters said immunogen for the first time. In many cases, a single administration of a vaccine is not sufficient to generate the number of long-lasting immune cells which is required for effective protection against said diseases or disorder. Consequently, repeated challenge with a biological preparation specific for a specific disease is required in order to establish lasting and protective immunity against said disease or to cure a given disease. An administration regimen comprising the repeated administration of a vaccine directed against the same disease is referred to in the present application as “prime-boost vaccination regimen”. A prime-boost vaccination regimen may involve at least two administrations of a vaccine or vaccine composition directed against a specific pathogen, group of pathogens or diseases. The first administration of the vaccine is referred to as “priming” and any subsequent administration of the same vaccine or a vaccine directed against the same pathogen as the first vaccine is referred to as “boosting”. Preferably, 1 to 10, 2 to 9, 2 to 8, 2 to 7, 2 to 6, 2 to 5 boost applications are used. The period of time between prime and boost is, preferably, 1 week, 2 weeks, 4 weeks, 6 weeks or 8 weeks. More preferably, it is 4 weeks or 8 weeks. If more than one boost is performed, the subsequent boost is, preferably, administered 1 week, 2 weeks, 4 weeks, 6 weeks or 8 weeks after the preceding boost. More preferably, the interval between any two boosts is 4 weeks or 8 weeks. Prime-boost vaccination regimens may be homologous or heterologous. In homologous prime-boost regimens both the priming and the at least one boosting is performed using the same means of administration of the antigenic protein or antigenic fragment thereof, i.e. priming and boosting are performed using a polypeptide or priming and boosting are performed using a nucleic acid construct comprised by the same vector. A heterologous prime-boosting regimen involves the use of different means for priming and for boosting the immune response.

In cases where a particular high antibody titer is required the boosting regimen following the initial prime-boost application may be continued for the duration of treatment. For preventing the diseases of the present invention, the boosting regimen may be continued as a life-long boosting regimen for the treated subjects to prevent eventual onset of the disease.

Two or more antigenic proteins or antigenic fragments thereof are “immunologically identical” if they are recognized by the same antibody, T-cell or B-cell. The recognition of two or more immunogenic polypeptides by the same antibody, T-cell or B-cell is also known as “cross reactivity” of said antibody, T-cell or B-cell. Preferably, the recognition of two or more immunologically identical polypeptides by the same antibody, T-cell or B-cell is due to the presence of identical or similar epitopes in all polypeptides. Similar epitopes share enough structural and/or charge characteristics to be bound by the Fab region of the same antibody or B-cell receptor or by the V region of the same T-cell receptor. The binding characteristics of an antibody, T-cell receptor or B-cell receptor are, preferably, defined by the binding affinity of the receptor to the epitope in question. Two immunogenic polypeptides are “immunologically identical” as understood by the present application if the affinity constant of polypeptide with the lower affinity constant is at least 30%, at least 40%, at least 50%, at least 60%, at least 70%, at least 80%, at least 90%, at least 95% or at least 98% of the affinity constant of the polypeptide with the higher affinity constant. Methods for determining the binding affinity of a polypeptide to a target such as equilibrium dialysis, enzyme linked immunosorbent assay (ELISA) or surface plasmon resonance are well known in the art. Preferably, two or more “immunologically identical” polypeptides comprise at least one identical epitope. The strongest vaccination effects can usually be obtained, if the immunogenic polypeptides comprise identical epitopes or if they have an identical amino acid sequence. The term “vaccine” refers to a biological preparation, typically a pharmaceutical, which improves immunity to a specific disease. Said preparation may comprise one or more disease specific immunogens suitable for eliciting an immune response. In the context of the present invention, said compound may be a polypeptide which is substantially identical or immunologically identical to a polypeptide as specified below comprising the specified dipeptide repeat (DPR). Alternatively, the vaccine may comprise a nucleic acid construct which encodes an immunogenic polypeptide which is substantially identical or immunologically identical to a polypeptide comprising the specified dipeptide repeat. In the latter case, it is preferred that the polypeptide is expressed in the individual treated with the vaccine. In the context of the present in invention said nucleic acid construct may be a vector.

As used herein, the term “vector” refers to a protein or a polynucleotide or a mixture thereof which is capable of being introduced or of introducing the proteins and/or nucleic acid comprised therein into a cell. Moreover, the term “vector” refers to at least one polynucleotide formulated with a preparation of liposomes or lipid nanoparticles which is capable of transfecting a cell with the at least one polynucleotide as described, e.g. by Geall et al., 2012. In addition to the polynucleotide encoding the gene of interest, additional polynucleotides and/or polypeptides may be introduced into the cell. The addition of further polynucleotides and/or polypeptides is especially preferred if said additional polynucleotides and/or polypeptides are required to introduce the nucleic acid construct into the cell or if the introduction of additional polynucleotides and/or polypeptides increases the expression of the immunogenic polypeptide encoded by the nucleic acid construct of the present invention.

In the context of the present invention it is preferred that the genes of interest encoded by the introduced polynucleotide are expressed within the cell upon introduction of the vector or vectors. Examples of suitable vectors include but are not limited to plasmids, cosmids, phages, viruses or artificial chromosomes.

Examples of a disease include but are not limited to neurological disorders, inflammatory diseases, infectious diseases, cutaneous conditions, endocrine diseases, intestinal diseases, genetic disorders, autoimmune diseases, traumatic diseases, joint diseases, and various types of cancer.

“Neurological disorders” refers to any disorder of the nervous system wherein structural, biochemical or electrical abnormalities occur in the brain, the spinal cord or other nerves which affect a range of symptoms including but not limited to paralysis, muscle weakness, poor coordination, loss of sensation, seizures, confusion, pain and altered levels of consciousness. Examples of neurological disorders include but are not limited to damage of the brain or individual parts of the brain (e.g. damage of the prefrontal cortex, frontal lobe, parietal lobe, temporal lobe, occipital lobe, cerebellum, hippocampus, brain stem, limbic system), dysfunction of the brain or individual parts of the brain (e.g. aphasia, dysarthria, apraxia, agnosia, amnesia, ataxia), or inflammation of the brain (e.g. encephalitis, viral encephalitis, cavernous sinus thrombosis, brain abscess, amoebic). “Neurodegenerative diseases” such as Alzheimer's, Parkinson's disease, Huntington's disease, spinocerebellar ataxia (SCA), amyotrophic lateral sclerosis (ALS), frontotemporal dementia (FTD), which is often called frontotemporal lobar degeneration (FTLD) by neuropathologists and is used interchangeably with FTD herein. Some patients fulfill the diagnostic criteria for both diseases and are thus diagnosed with amyotrophic lateral sclerosis-frontotemporal dementia (ALS-FTD, also called ALS-FTLD). The clinical and pathological terms FTD and FTLD (and ALS-FTD and ALS-FTLD) are used synonymously in this application. Neurological disorders also include spinal cord disorders (e.g. syringomyelia, syringobulbia, Morvan's syndrome, Vascular myelopathy, Foix-Alajouanine syndrome, spinal cord compression) and spinal cord inflammation (e.g. myelitis, poliomyelitis, demyelinating disease, transverse myelitis, tropical spastic paraparesis, epidural abscess), central and/or peripheral neuropathy, cranial nerve disorders (e.g. trigeminal neuralgia), movement disorders of the central and/or peripheral nervous system (e.g. Parkinson's disease, ALS, Tourette's Syndrome, multiple sclerosis), sleep disorders (e.g. insomnia, hypersomnia, sleep apnea, narcolepsy, cataplexy, Kleine-Levin, circadian rhythm sleep disorder, advanced sleep phase disorder, delayed sleep phase disorder), headache (e.g. migraine, cluster, tension), neuropsychiatric illnesses, delirium, dementia (e.g. Alzheimer's disease, vascular dementia, FTD, semantic dementia and dementia with Lewy bodies), stroke (e.g. MCA, ACA, PCA, Foville's, Millard-Gubler, Lateral medullary, Weber's, Lacunar stroke), tumors (e.g. gliomas, meningiomas, pituitary adenomas, nerve sheath tumors), complex regional pain syndrome, and motor neuron diseases (MND) (e.g. ALS, primary lateral sclerosis (PLS), progressive muscular atrophy (PMA), progressive bulbar palsy (PBP), pseudobulbar palsy).

“Symptoms” of a disease are implication of the disease noticeable by a cell, tissue, organ or individual having such disease and include but are not limited to pain, weakness, tenderness, strain, stiffness, and spasm of the cell, tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms.

The term “indicator” or “biomarker” are used interchangeably herein. In the context of the present invention, an “indicator” can be defined as a substance within a biological system that is used as an indicator of a biological state of said system. In the art, the term “biomarker” is sometimes also applied to means for the detection of said endogenous substances (e.g. antibodies, nucleic acid probes, imaging systems). In the context of present invention, however, the term “biomarker” shall be only applied for the substance, not for the detection means. Thus, biomarkers can be any kind of molecule present in a living organism, such as a nucleic acid (DNA, mRNA, miRNA, rRNA etc.), a protein (cell surface receptor, cytosolic protein etc.), a metabolite or hormone (blood sugar, insulin, estrogen, etc.), a molecule characteristic of a certain modification of another molecule (e.g. sugar moieties or phosphoryl residues on proteins, methyl-residues on genomic DNA, expansion of nucleotide repeats) or a substance that has been internalized by the organism or a metabolite of such a substance. Accordingly, a disease or disorder may be characterized by the presence or absence, increase or decrease of such an indicator. Said indicator of a disease may or may not cause the disease.

Indicators of the presence and/or progression of a disease include “genetic markers” such as nucleotide repeat, VNTRs (variable number tandem repeat; e.g. STR (Short tandem repeat), AFLP (amplified fragment length polymorphism), SSR (Simple sequence repeat), MLVA), SSLP (Simple sequence length polymorphism), RFLP (restriction fragment length polymorphism), RAPD (random amplification of polymorphic DNA), SNP (single nucleotide polymorphism), SFP (single feature polymorphism), DArT (diversity arrays technology), RAD markers (restriction site associated DNA markers).

The term “nucleotide repeat” refers to a location in the genome wherein a short nucleotide sequence forms repeating sequences of 2, 3, 4, 5, or 6 nucleotides, i.e. dinucleotides, trinucleotides, tetranucleotides, pentanucleotides, and hexanucleotides. In a healthy state the number of repetitions of the repeat may vary between 1 and 50, i.e. 1, 2, 3, 4, 5, 6, 7, 8, 9, 10, 15, 20, 25, 30, 35, 40, 45, 50. However, in a diseased state these repeats may be expanded to be repeated far more often than in a healthy state, e.g. they may be expanded 30 to 40 times, leading to several hundred repetition of the repeat, i.e. 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000, or even more repetitions. Accordingly, the expansion level of dinucleotides, trinucleotides, tetranucleotides, pentanucleotides, or hexanucleotides may be an indicator (and/or the cause) of a disease such as e.g. a neurological disease, in particular a neurogenerative disease. Exemplified, the expansion of trinucleotide repeats (CAG)ₙ and of the reverse complement (CTG)ₙ in the complementary strand is associated with Huntington's disease, the expansion of pentanucleotide repeats (ATTCT)ₙ and of the reverse complement (AGAAT)ₙ in the complementary strand is associated with spinocerebellar ataxia SCA10, whilst the expansion of hexanucleotides (GGGGCC)ₙ and of the reverse complement (CCCCGG)ₙ in the complementary strand is associated with a subset of ALS, FTD, and FTD-ALS and the expansion of the hexanucleotides ((GGCCTG)ₙ and of the reverse complement (CAGGCC)ₙ in the complementary strand is associated with spinocerebellar ataxia SCA36. In principle, nucleotide repeats may be present in the exon or intron of a gene and may or may not be transcribed and may or may not be translated into a peptide or polypeptide.

The neurodegenerative diseases ALS, FTD and ALS-FTD are characterized in that the hexanucleotide (GGGGCC)ₙ repeats located upstream of the coding region of the C9orf72 gene are expanded. In a healthy state the number “n” of the GGGGCC-repeats may vary between 1 and 19, i.e. 1, 2, 3, 4, 5, 6, 7, 8, 9, 10, 15, and 19. In a diseased state the number “n” of these repeats may be expanded to be repeated far more often than in a healthy state, e.g. typically they may be expanded much more than 40 times. Segregation of 40-50 repeat expansion with disease has been shown in some families (Gijselinck et al., Molecular Psychiatry 2016). Intermediate size repeats with 20-45 repeats are rare and likely benign (Kaivola et al., Neurobiol Aging. 2019). Thus, in a diseased state the expansion of the hexanucleotide repeats may lead to several hundred repetitions of the hexanucleotide repeat, i.e. 30, 35, 40, 45, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1,000, 2,000, 3,000, 4,000, 5,000, 6,000, 7,000, 8,000, 9,000, and 10,000 repeats, or more. The exact number of repeats cannot be determined with standard sequencing techniques and long-read sequencing has only reported for few cases (Ebbert et al., Mol Neurodegener. 2018). Transcription of the genomic GGGGCC-repeats and of the GGCCCC-repeats in the complementary DNA strand (also termed antisense strand) leads to RNA transcription products which comprise either GGGGCC- or GGCCCC-repeats with the above indicated numbers. Surprisingly, translation of these transcripts is initiated from non-ATG codons in most reading frames. The sense transcript is translated in all three reading frames (poly-(Gly-Ala), poly-(Gly-Pro) and poly-(Gly-Arg)). Thus 6 reading frames exist, but two result in DPRs that are identical apart from potential N- and C-terminal extensions, namely poly-(Gly-Pro) DPR. Accordingly, the five different possible DPRs are the following: poly-(Gly-Ala), poly-(Gly-Pro), poly-(Gly-Arg), poly-(Ala-Pro) and poly-(Pro-Arg). In particular the poly-GA, poly-GP and poly-AP polypeptides are highly hydrophobic. Upon individual expression from synthetic genes only poly-GA forms abundant cytoplasmic aggregates in an intracellular environment.

The genomic sequence of the region surrounding the hexanucleotide repeat of the C9orf72 gene reads as follows in the GRCh38/hg38 reference genome sequence from NCBI (SEQ ID No: 004)

acgtaacctacggtgtcccgctaggaaagagaggtgcgtcaaacagcg
acaagttccgcccacgtaaaagatgacgcttggtgtgtcagccgtccc
tgctgcccggttgcttctatttgggggcggggtctagcaagagcaggt
gtgggtttaggaggtgtgtgtttttgtttttcccaccctctctcccca
ctacttgctctcacagtactcgctgagggtgaacaagaaaagacctga
taaagattaaccagaagaaaacaaggagggaaacaaccgcagcctgta
gcaagctctggaactcaggagtcgcgcgcta[GGGGCC]ₙggggcgtg
gtcggggcgggcccgggggcgggcccggggcggggctgcggttgcggt
gcctgcgcccgcggcggcggaggcgcaggcggtggcgagtgggtgagt
gaggaggcggcatcctggcgggtggctgtttggggttcggctgccggg
aagaggcgcgggtagaagcgggggctctcctcagagctcgacgcattt
ttactttccctctcatttctctgaccgaagctgggtgtcgggctttcg
cctctagcgactggtggaattgcctgcatccgggccccgggcttcccg
gcggcggcggcggcggcggcggcgcagggacaagggatggggatctgg
cctatccttgctttcccgccctcagtacccgagctgtctccttcccgg
ggacccgctgggagcgctgccgctgcgggctcgagaaaagggagcctc
gggtactgagaggcctcgcctgggggaaggccggagggtgggcggcgc
gcggcttctgcggaccaagtcggggttcgctaggaacccgagacggtc
cctgccggcgaggagatcatgcggg,

wherein “n” has the above outlined preferred and most preferred meanings.

5′ and 3′ of the genomic repeat region further polypeptide encoding regions termed “non-repetitive regions” are located. The sequence of the non-repetitive regions depends on the genomic location of the hexanucleotide repeat region. Accordingly, the DPRs may further comprise additional amino acid sequences at its N- and/or C-Terminus. A polypeptide comprising a dipeptide repeat region further comprises additional C-terminal amino acid sequence encoded by the genomic region 3′ of the repeat region. The exact start or initiation site of translation is under debate. In the case of RAN-translation the start site may be within the repeat region or within the flanking region. Initiation from a non-canonical start-codon may also lead to an N-terminal flanking region with the largest N-terminal region extending to the first in-frame stop-codon at the 5′ end of the RNA. In addition, frameshifts within the repeat may occur during translation. Depending on the transcription initiation of the RAN-translated mRNA encoding the respective DPR, the DPR may also comprise N-terminal amino acid sequence encoded by the genomic region 5′ of the repeat region. The length of the respective non-repetitive C-terminal amino acid sequence depends on the position of the first STOP codon in the respective reading frame that gives rise to the DPR. Thus, while two diseases each characterized by expansion of (GGGGCC)ₙ repeats in different genomic regions may lead to identical DPRs, the C- and/or N-terminal non-repetitive regions comprised in the polypeptide will vary depending on the genomic context and will allow distinguishing between two diseases characterized by expansion of genomic hexanucleotide repeats. For each disease characterized by hexanucleotide repeats the skilled person is able to determine the respective 5′ and/or 3′ genomic region and, thus, the amino acid sequences that may be comprised N- and/or C-terminally of the DPR. Known polymorphisms in the 3′ region of the GGGGCC repeat may additionally cause frame shifts within the 3′ region.

Amino acid sequences that may be comprised at the C-terminus of the DPRs characteristic for ALS, FTD, or ALS-FTD include for
(i) the (Gly-Ala) repeat: WSGRARGRARGGAAVAVPAPAAAEAQAVASG (SEQ ID NO: 005);
(ii) the (Gly-Pro) repeat: GRGRGGPGGGPGAGLRLRCLRPRRRRRRWRVGE (SEQ ID NO: 006) or no C-terminal sequence as the antisense GP-repeat is directly followed by a STOP-codon;
(iii) the (Gly-Arg) repeat: GVVGAGPGAGPGRGCGCGACARGGGGAGGGEWVSEEA ASWRVAVWGSAAGKRRG (SEQ ID NO: 007);
(iv) the (Ala-Pro) repeat: SARLLSSRACYRLRLFPSLFSSG (SEQ ID NO: 008);
(v) the (Pro-Arg) repeat: PLARDS (SEQ ID NO: 009).
   Based on the nearest upstream stop codon the amino acid sequences that may be comprised at the N-terminus of the DPRs characteristic for ALS, FTD, or ALS-FTD include for
(i) the (Gly-Ala) repeat: QALELRSRAL (SEQ ID NO: 010);
(ii) the (Gly-Pro) repeat: GRESKEEARSPSLVPAPPPPPPPPGSPGPGCRQFHQSLEAK ARHPASVREMRGKVKMRRALRRAPASTRASSRQPNPKQPPARMPPPHSPTR HR LRLRRRGRRHRNRSPAPGPPPGPPRPRP (SEQ ID NO: 011);
(iii) the (Gly-Arg) repeat: RLTRRKQGGKQPQPVASSGTQESRAR (SEQ ID NO: 012);
(iv) the (Ala-Pro) repeat: GEPPLLPAPLPGSRTPNSHPPGCRLLTHPLATACASAAAG AGTATAAPPRARPRARPDH (SEQ ID NO: 013);
(v) the (Pro-Arg) repeat: SPRRQGPSRVPSEPRLGPQKPRAAHPPAFPQARPLSTRGSL FSSPQRQRSQRVPGKETARVLRAGKQARMQAIPPVARGESPTPSFGQRNERES KNASSSEESPRFYPRLFPAAEPQTATRQDAASSLTHSPPPAPPPPRAQAPQPQPRPGP APGPAPTT (SEQ ID NO: 014). The exact initiation point within this sequence is under debate. Regular AUG initiation is also possible from the underlined codon Poly-PR may also initiate at a regular AUG start codon.

Accordingly, the polypeptide resulting from the hexanucleotide repeat in the C9orf72 gene in sense and/or anti sense direction may have one of the following sequences from stop codon to stop:

| | |
|---|---|
| (i) | SEQ ID NO: 015: qalelrsralGA[GA]ₘGAwsgrargr |
| | arggaavavpapaaaeaqavasg |
| (ii) | SEQ ID NO: 016: GP[GP]ₒGPgrgrggpgggpgaglrlr |
| | clrprrrrrrrwrvge |
| (iii) | SEQ ID NO: 017: greskeearspslvpappppppppgs |
| | pgpgcrqfhqsleakarhpasvremrgkvkmrralrrapast |
| | rassrqpnpkqpparmppphsptrhrlrlrrrgrrhrnrspa |
| | pgpppgpprprpGP[GP]ₒGP |
| (iv) | SEQ ID NO: 018: rltrrkqggkqpqpvassgtqesrar |
| | GR[GR]ₚGRgvvgagpgagpgrgcgcgacarggggagggewv |
| | seeaaswrvavwgsaagkrrg |
| (v) | SEQ ID NO: 019: geppllpaplpgsrtpnshppgcrll |
| | thplatacasaaagagtataapprarprarpdhAP[AP]_{q}AP |
| | sarllssracyrlrlfpslfssg |
| (vi) | SEQ ID NO: 020: sprrqgpsrvpseprlgpqkpraahp |
| | pafpgarplstrgslfsspqrqrsqrvpgketarvlragkqg |
| | rgqipipcpcaaaaaaaagkpgarmqaippvargesptpsfg |
| | qrneresknassseesprfyprlfpaaepqtatrqdaasslt |
| | hspppappppraqapqpqprpgpapgpapttPR[PR]ᵣPRpl |
| | ards. |

Wherein m is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; “o” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; “p” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; “q” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; “r” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5000 or more; “s” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; “t” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; “u” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; “w” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; “x” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more; and “y” is an integer of 10 or more, preferably 15, 20 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,500, 2,000, 2,500, 3,000, 3,500, 4,000, 4,500, 5,000 or more.

It is preferred that the C-terminus of the amino acid sequences according to SEQ ID NO: 010 to 014 is connected via a peptide bond with the N-terminus of the respectively indicated DPR. Preferably, the DPR comprises at least three contiguous, more preferably at least five contiguous amino acids, more preferably at least ten contiguous amino acids of the amino acids according to SEQ ID NO: 010 to 014, most preferably the entire amino acid sequence.

The term “aggregates” as used herein refers to the intra- or extracellular accumulation of peptides, polypeptides (e.g. DPR) or proteins.

The terms “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, lymphatic fluid, cerebrospinal fluid, meningeal fluid, glandular fluid, fine needle aspirate, spinal fluid and other body fluids (urine, saliva), as well as biopsy sample or solid samples such as tissue extracts of the brain or spinal cord. Further examples of samples include cell cultures (e.g. patient derived lymphoblasts) or tissue cultures.

Analysis of a sample may be accomplished on a visual or chemical basis. Visual analysis includes but is not limited to microscopic imaging or radiographic scanning of a tissue, organ or individual allowing for the evaluation of a sample. Chemical analysis includes but is not limited to the detection of the presence or absence of specific indicators or the detection of the alterations in their amount or level.

The twit “ligand” as used herein refers to any substance or compound that is able to specifically interact with, e.g. to specifically bind to or to form a complex with, the specified molecule, e.g. a polypeptide of the invention comprising or consisting of a dipeptide repeat. Preferred ligands are antibodies or antigen-binding fragments thereof. The term “inhibitor” refers to a substance, e.g. a ligand, which blocks the action of another compound, i.e. a receptor molecule. Typically, inhibitors act by binding to the active site of the receptor molecule, or by interacting with unique binding sites not normally involved in the regulation of the activity of the receptor molecule. The activity of the inhibitor may be reversible or irreversible depending on the longevity of the interaction of the inhibitor-receptor molecule complex. Examples for inhibitors include but are not limited to nucleic acid molecules, such as siRNAs or miRNAs, or proteins such as transcription factors, immunoglobulin molecules, antibodies, antibody-like proteins, peptidomimetics, hormones, cytokines, growth factors, or neurotransmitters.

The terms “specific binding” or “specifically binding” to an antigen refers to the ability of a ligand to bind to an antigenic determinant of an antigen with high affinity. In that context “high affinity” means that the K_{d} for the interaction is below 1×10⁻⁵ M, preferably below 1×10⁻⁶ M, more preferably below 1×10⁻⁷, even more preferably below 1×10⁻⁸ M and most preferably below 1×10⁻⁹ M.

The terms “pharmaceutical”, “pharmaceutical composition”, “medicament”, “medical agent”, “agent” and “drug” are used interchangeably herein referring to a substance and/or a combination of substances being used for the identification, prevention or treatment of a tissue status or disease.

“Pharmaceutically acceptable” means approved by a regulatory agency of the Federal or a state government or listed in the U.S. Pharmacopeia or other generally recognized pharmacopeia for use in animals, and more particularly in humans.

The term “active ingredient” refers to the substance in a pharmaceutical composition or formulation that is biologically active, i.e. that provides pharmaceutical value. A pharmaceutical composition may comprise one or more active ingredients which may act in conjunction with or independently of each other. The active ingredient can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as but not limited to those derived from sodium, potassium, ammonium, calcium, ferric hydroxides, isopropylamine, triethylamine, 2-ethylamino ethanol, histidine, procaine, and the like.

The term “carrier”, as used herein, refers to a pharmacologically inactive substance such as but not limited to a diluent, excipient, or vehicle with which the therapeutically active ingredient is administered. Such pharmaceutical carriers can be liquid or solid. Liquid carrier include but are not limited to sterile liquids, such as saline solutions in water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

Suitable pharmaceutical “excipients” include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The term “carrier protein” as used herein refers to an immunogenic protein that is used to increase the response of the immune system to an otherwise not sufficiently immunogenic compound, such as another protein/peptide. Carrier proteins are in particular useful for small compounds that have a very limited number of possible epitopes on which an immune response can be directed or compounds that have a very low immunogenicity. Preferred carrier proteins have a high compound to carrier ratio by allowing the coupling of numerous compounds to a single carrier protein in order to increase the immunogenicity of a carrier protein antigen complex. Various methods for chemical crosslinking of peptides to carrier proteins are known in the arts and typically involve reactive sulfhydryl and/or amino groups. Many of these systems are commercially available (e.g. Imject™ from ThermoFisher Scientific). Most commonly chemical crosslinking is done using a sulfhydryl group in the antigen, which can be introduced by addition of a cysteine residue to the antigenic peptide, e.g. C-(GA)₁₀, or via a primary amino group (e.g. Imject™ Maleimide and Imject™ EDC products from ThermoFisher Scientific).

The term “adjuvant” refers to agents that augment, stimulate, activate, potentiate, or modulate the immune response to the active ingredient of the composition at either the cellular or humoral level, e.g. immunologic adjuvants stimulate or modulate the response of the immune system to the actual antigen, but have no immunological effect themselves. Examples of such adjuvants include but are not limited to inorganic adjuvants (e.g. inorganic metal salts such as aluminum phosphate or aluminum hydroxide such as Alhydrogel® often referred to as alum), organic adjuvants (e.g. saponins or squalene), oil-based adjuvants (e.g. Freund's complete adjuvant and Freund's incomplete adjuvant (preferably Montanide ISA 51, Montanide ISA71 VG and Montanide ISA206), cytokines (e.g. IL-1(3, IL-2, IL-7, IL-12, EL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A (MPL), or muramyl peptides), synthetic adjuvants (e.g. non-ionic block copolymers, muramyl peptide analogues, or synthetic lipid A), or polynucleotides adjuvants (e.g CpG oligodeoxynucleotides, preferably synthetic CpG ODNs). QS-21 is a saponin extracted from Quillaja saponaria. Mf59 is an oil-in-water emulsion comprising squalene, polysorbate 80 and sorbitantrioleat. AS03 is an oil-in-water emulsion comprising squalene, polysorbate 80 and α-tocopherol. AS01 is a combination of liposomes, MPL and QS21. AS02 is a combination of oil-in-water emulsion, MPL and QS21. AS04 is a complex of MPL and aluminum hydroxide or aluminum phosphate. IC31 is a combination of a KLK peptide with the oligodesoxynucleotide ODN1. Hiltonol (Poly-ICLC) is a synthetic complex of carboxymethylcellulose, polyinosinic-polycytidylic acid, and poly-L-lysine double-stranded RNA. Preferred adjuvants are Freund's incomplete adjuvant (in particular Montanide ISA 51, Montanide ISA71 VG and Montanide ISA206), Hiltonol, Alum and CpG ODN (in particular CpG ODN 2006 and CpG ODN 1668).

Pharmaceuticals are administered via a route of administration suitable to the case. Administration routes include but are not limited to intranasal administration, intramuscular administration, subcutaneous administration, oral administration, and topical administration.

An “intranasal administration” is the administration of a pharmaceutical to the mucosa of the complete respiratory tract including the lung. Typically, the pharmaceutical is administered to the mucosa of the nose. An intranasal administration is achieved by means of instillation, spray or aerosol. Preferably, said administration does not involve perforation of the mucosa by mechanical means such as a needle.

The term “intramuscular administration” refers to the injection of a pharmaceutical into any muscle of an individual or a patient. Preferred intramuscular injections are administered into the deltoid, vastus lateralis or the ventrogluteal and dorsogluteal areas.

The term “subcutaneous administration” refers to the injection of a pharmaceutical into the hypodermis.

The term “oral administration” refers to the administration of a pharmaceutical via the mouth to the gastric system.

A “topical administration” is the administration of a pharmaceutical to any part of the skin without penetrating the skin with a needle or a comparable device. The pharmaceutical may also be administered topically to the mucosa of the mouth, nose, genital region and rectum.

The terms “presymptomatic” and “prodromal” refer to very early stages of a disease. In the presymptomatic stage the subject is without any symptoms of the disease. Preferred examples of presymptomatic stages are subjects with an increased risk of developing a certain disease that have not yet displayed any symptoms of this disease. In the prodromal stage of a disease early symptoms of a disease are already present although those symptoms are not diagnostically specific symptoms of this disease and may occur in other diseases as well.

### Embodiments

In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect the present invention provides an immunogen for use in preventing or treating familial frontotemporal dementia (FTD), amyotrophic lateral sclerosis (ALS) or combined amyotrophic lateral sclerosis-frontotemporal dementia (ALS-FTD) in patients with C9orf72 repeat expansion comprising or consisting of a polypeptide consisting of dipeptide-repeats with a sequence selected from the group consisting of (Gly-Ala)ₐ, (Gly-Pro)ₐ, (Gly-Arg)ₐ, (Pro-Ala)ₐ and (Pro-Arg)ₐ, preferably (Gly-Ala)ₐ wherein a is an integer of 4 to 100. In a preferred embodiment of the first aspect of the invention a is an integer of 4 to 25 or an integer of 7 to 15. In a more preferred embodiment of the first aspect of the invention a is an integer of 8 to 12.

The immunogen of the first aspect of the present invention is intended for use in immunization. The immunogen is intended to initiate an immune response against the indicated dipeptide-repeat proteins in order to prevent the onset of FTD and/or ALS or in case that the disease has manifested itself already in the treatment of said diseases. Without wishing to be bound by theory, the inventors believe that the immune response initiated by the immunogen is directed against the dipeptide-repeat proteins that can form aggregates which are an underlying cause of FTD and/or ALS in C9orf72 mutation carriers. Preventing the (further) formation of such aggregates is believed to prevent further aggravation of the disease and/or complete remission of the DPR aggregates and may stop the disease cascade. In particular, the inventors showed that poly-GA expression causes non-cell-autonomous cytoplasmic mislocalization of TDP-43 in neighboring cells, which is prevented by adding monoclonal anti-GA antibodies to the co-culture assay (see FIGS. 6 and 7). Using a strongly immunogenic antigen formulation, polyclonal antibodies resulting from active immunization against poly-GA may have similar effects. Surprisingly, the immunogen of the first aspect of the invention results in unexpectedly high antibody titers in excess of 400 μg/ml (see FIG. 1B and FIG. 10A). These high levels are beneficial for the intended use because antibodies have to cross the blood brain barrier. Indeed, antibody levels in the CSF reach around −0.1 to 5 μg/ml (median level of 0.2219 μg/ml; FIG. 10C), which is comparable to the 1 μg/ml monoclonal antibodies inducing beneficial effects on poly-GA levels and TDP-43 mislocalization in cellular models (FIG. 7 and Khosravi et al, EMBO J 2020, Zhou et al, EMBO Mol Med 2017).

In one embodiment the dipeptide-repeats with a sequence selected from the group consisting of (Gly-Ala)ₐ, (Gly-Pro)ₐ, (Gly-Arg)ₐ, (Pro-Ala)ₐ and (Pro-Arg)ₐ, preferably (Gly-Ala)ₐ are aggregated.

The immunogen of the first aspect of the invention may further comprise amino acid sequences N- and/or C-terminally of the polypeptide consisting of the dipeptide repeat protein.

Preferred sequences would include carrier proteins, spacers and sequences characteristic for ALS, FTD, or ALS-FTD, preferably the sequences of SEQ ID NOs 005 to 014.

In a preferred embodiment of the first aspect of the invention the polypeptide consists of (Gly-Ala)ₐ, wherein a is an integer of 4 to 100, preferably 4 to 25, preferably 7 to 15, more preferably 8 to 12, most preferably 9 to 11.

In a preferred embodiment of the first aspect of the invention the polypeptide further comprises at least one non-standard amino acid, preferably a non-proteinogenic amino acid, more preferably an amino acid selected from norleucine (Nle) and norvaline (Nva). In a preferred embodiment the non-standard amino acid is preceding the dipeptide-repeats. An exemplary polypeptide could have the sequence Gly-Nle-Gly-Ala-Gly-Ala-Gly-Ala-Gly-Ala-Gly-Ala-Gly-Ala-Gly-Ala-Gly-Ala-Gly-Ala-Gly-Ala. The advantage of using a non-standard amino acid is the possibility of quantifying the coupling efficacy of the peptide to an immunogenic carrier. The non-standard amino acids are therefore preferably used in immunogens comprising an immunogenic carrier. An exemplary polypeptide could have the sequence KLH-PEG-Gly-Nle-Gly-Ala-Gly-Ala-Gly-Ala-Gly-Ala-Gly-Ala-Gly-Ala-Gly-Ala-Gly-Ala-Gly-Ala-Gly-Ala.

In a preferred embodiment of the first aspect of the invention the immunogen for use is further comprising a carrier protein. Preferably said carrier protein is selected from tetanus toxoid (TT), HSP60, Concholepas concholepas hemocyanin (CCH), diphtheria toxin CRM197, diphtheria toxoid (DT), meningococcal outer membrane protein complex (OMPC), ovalbumin (OVA), keyhole limpet hemocyanin (KLH) or bovine serum albumin (BSA). More preferably said carrier protein is selected from tetanus toxoid, diphtheria toxoid, keyhole limpet hemocyanin and ovalbumin. More preferably, the carrier protein is OVA or KLH. Most preferably the carrier protein is KLH.

In a preferred embodiment the immunogen for use is expressed as a fusion protein with the carrier protein. A carrier protein is used to increase the response of the immune system to an otherwise not sufficiently immunogenic compound. This is in particular useful for small compounds that have a very limited number of possible epitopes on which an immune response can be directed. Preferred carrier proteins have a high compound to carrier ratio by allowing the coupling of numerous compounds to a single carrier protein.

In a preferred embodiment of the first aspect of the invention the carrier protein is non-covalently or covalently linked to the polypeptide. The linkage of the carrier protein can be direct or via a spacer introduced between immunogen and carrier protein. Preferably the carrier protein is linked by a spacer. A preferred spacer is a PEG spacer. More preferred is a PEG spacer containing 3 PEG subunits. Polyethylene glycol (PEG) is a polyether compound with the structure H—(O—CH2-CH2)ₙ-OH, wherein n is an integer indicating the number of PEG subunits. Preferred PEG spacers of the present invention have n=1 to 10, more preferably n=2 to 4, most preferably n=3 (i.e. 3 PEG subunits).

In a preferred embodiment of the first aspect of the invention the carrier protein is linked to the C-terminus of the polypeptide or the spacer, if present.

In a preferred embodiment of the first aspect of the invention the carrier protein is linked to the N-terminus of the polypeptide or the spacer, if present.

In a preferred embodiment of the first aspect of the invention the FTD, ALS and/or ALS-FTD prevented or treated is caused by the expansion of a (GGGGCC) hexanucleotide repeat upstream of C9orf72 coding region.

In a preferred embodiment of the first aspect of the invention the immunogen is used in presymptomatic carriers of the C9orf72 repeat expansion for prevention or treatment of a prodromal stage of FTD, ALS and ALS-FTD. The long prodromal stage of C9orf72 ALS/FTD provides the opportunity to apply very early or even presymptomatic treatment of known mutation carriers to achieve maximal efficiency of treatment.

In a further preferred embodiment of the first aspect of the invention the immunogen is used in presymptomatic carriers of the C9orf72 repeat expansion for prevention or treatment of FTD, ALS and ALS-FTD.

In a further preferred embodiment of the first aspect of the invention the immunogen is used in a prodromal stage of FTD, ALS and ALS-FTD in carriers of the C9orf72 repeat expansion for prevention or treatment of FTD, ALS and ALS-FTD.

In a further preferred embodiment of the first aspect of the invention the immunogen is used in a prodromal stage of FTD, ALS and ALS-FTD in carriers of the C9orf72 repeat expansion for treatment of the prodromal stage of FTD, ALS and ALS-FTD.

In a further preferred embodiment of the first aspect of the invention the FTD, ALS and/or ALS-FTD is in a prodromal stage.

In a further preferred embodiment of the first aspect of the invention the FTD, ALS and/or ALS-FTD is in a presymptomatic stage.

In a preferred embodiment the immunogen is used in a prime-boost regimen, consisting of at least one boost application. Preferably, dosing is 40 μg peptide coupled to a carrier protein (e.g. KLH or ovalbumin), preferably by a spacer such as 3 subunits of PEG. Preferred embodiments consist of 2 to 8, more preferably 2 to 6, most preferably 4 to 5 boost applications. The prime-boost regimen can be homologous or heterologous, preferably homologous. A heterologous prime-boost regimen would use different immunogens for prime and/or boost applications, wherein a homologous prime-boost regimen would use the same immunogen. The time between prime and/or boost application of the immunogen is 2 to 12 weeks, preferably 3 to 9 weeks, more preferably 4 to 5 weeks, most preferably 4 weeks.

In a preferred embodiment the immunogen is used in a continuous boost regimen after the initial prime-boost regimen. The continuous boost regimen is aimed at the maintenance of continuously high antibody titers. The mere presence of specific memory cells may not be sufficient to maintain sufficient immunogenic response throughout the life of a treated subject. The continuous boost regimen would result in continuously high antibody titers. Preferably, the continuous boost regimen is applied until the antibody titers have reached a stable plateau and a boost application does not increase the antibody titers any further. A further boost in the continuous boost regimen is applied once the antibody titer drops 10%, 15% or 20% below the level of the plateau phase. Preferably, boosting is required at least twice per year, preferably 4 to 6 times per year to maintain high titers.

In a preferred embodiment the continuous boost regimen includes life-long boost applications. The time between the boost applications is 1 to 12 months, preferably 2 to 4 months. A preferred regimen is boosting every other month for life.

In a preferred embodiment, the boosting regimen is determined by a physician depending on clinical parameters and the anti-GA antibody titer, which needs to be checked regularly, for example using an assay as shown in FIG. 1B. Biomarkers detecting poly-GA load in CSF or by imaging could also be used to guide the therapy, but have not been developed yet.

In a second aspect the present invention provides an immunogenic composition comprising or consisting of the immunogen of the first aspect of the invention, a pharmaceutically acceptable carrier and/or suitable excipient(s).

In a preferred embodiment of the second aspect of the invention the immunogenic composition is further comprising an adjuvant. The adjuvant usably to exercise the invention includes any adjuvant known in the art that boosts the immune response to the immunogenic composition and/or results in a longer-lasting immunity without resulting in an immune response by themselves. Preferred adjuvants allow the reduction of the immunogenic composition to be applied for a sufficient immune response.

Preferably the immunogenic composition of the second aspect of the invention is comprising an adjuvant selected from:
incomplete Freund adjuvant (preferably Montanide ISA 51) or related formulations (preferably Montanide ISA71 VG and Montanide ISA206),
CpG oligodeoxynucleotides (ODN), preferably CpG ODN 2006 or CpG ODN 1668,
inorganic adjuvants, preferably aluminium hydroxide (preferably Alhydrogel) or aluminium phosphate;
organic adjuvants, preferably saponins or squalene, more preferably QS-21;
oil in water emulsions, preferably MF59 or AS03;
cytokines, preferably IL-1β, IL-2, IL-7, IL-12, IL-18, GM-CFS, or INF-γ;
particulate adjuvants, preferably ISCOMS, liposomes, or biodegradable microspheres;
virosomes;
bacterial adjuvants, preferably monophosphoryl lipid A, or muramyl peptides;
synthetic adjuvants, preferably non-ionic block copolymers, muramyl peptide analogues, or synthetic lipid A; and
combinations thereof, preferably AS01, AS02, AS04 or IC31.

In a more preferred embodiment the adjuvant is selected from: Freund's incomplete adjuvant (preferably Montanide ISA 51, Montanide ISA71 VG and Montanide ISA206), Hiltonol, inorganic adjuvants (preferably aluminum hydroxide or aluminum phosphate) and CpG ODN (preferably CpG ODN 2006 and CpG ODN 1668).

In a preferred embodiment the adjuvant is Freund's incomplete adjuvant (preferably Montanide ISA 51, Montanide ISA71 VG and Montanide ISA206) in combination with CpG ODN (preferably CpG ODN 2006 and CpG ODN 1668).

In a third aspect the present invention provides an immunogenic composition according to the second aspect of the invention for use in preventing or treating FTD, ALS and/or ALS-FTD caused by the expansion of a (GGGGCC) hexanucleotide repeat upstream of C9orf72 coding region.

In a further preferred embodiment of the third aspect of the invention the FTD, ALS and/or ALS-FTD is in a prodromal stage in carriers of the C9orf72 repeat expansion.

In a further preferred embodiment of the third aspect of the invention the FTD, ALS and/or ALS-FTD is in a presymptomatic stage in carriers of the C9orf72 repeat expansion.

In a fourth aspect the present invention provides a kit comprising the immunogen according to the first aspect of the invention, or the immunogenic composition according to the second aspect of the invention; and optionally at least one adjuvant. Optionally further comprising a container, and/or a data carrier, preferably comprising instructions for one or more of the first to third and fifth aspect of the present invention.

Preferably adjuvants are selected from:
CpG oligodeoxynucleotides, preferably CpG 2006 or CpG 1668,
inorganic adjuvants, preferably aluminum hydroxide or aluminum phosphate;
organic adjuvants, preferably saponins or squalene, more preferably QS-21;
oil in water emulsions, preferably MF59 or AS03;
cytokines, preferably IL-1β, IL-2, IL-7, IL-12, IL-18, GM-CFS, or INF-γ;
particulate adjuvants, preferably ISCOMS, liposomes, or biodegradable microspheres;
virosomes;
bacterial adjuvants, preferably monophosphoryl lipid A, or muramyl peptides;
synthetic adjuvants, preferably non-ionic block copolymers, muramyl peptide analogues, or synthetic lipid A; and
combinations thereof, preferably AS01, AS02, AS04 or IC31.

In a fifth aspect the present invention provides a method of treatment or prevention of FTD, ALS and/or ALS-FTD in a subject, wherein the method comprises administering an immunogen according to the first aspect of the invention or an immunogenic composition according to the second aspect of the invention.

In a preferred embodiment of the fifth aspect of the invention the FTD and/or ALS is caused by the expansion of a (GGGGCC) hexanucleotide repeat in the C9orf72 gene upstream of coding region.

In a further preferred embodiment of the fifth aspect of the invention the FTD, ALS and/or ALS-FTD is in a prodromal stage in carriers of the C9orf72 repeat expansion.

In a further preferred embodiment of the fifth aspect of the invention the FTD, ALS and/or ALS-FTD is in a presymptomatic stage in carriers of the C9orf72 repeat expansion.

In a sixth aspect the present invention provides a nucleic acid encoding the immunogen according to the first aspect of the invention.

The nucleic acid encoding said immunogen refers only to immunogens consisting of polypeptides.

In a seventh aspect the present invention provides a vector comprising the nucleic acid of the sixth aspect of the invention, preferably wherein the vector is a viral vector.

In an eight aspect the present invention provides an immunogen comprising or consisting of
a polypeptide comprising or consisting of dipeptide-repeats with a sequence selected from the group consisting of (Gly-Ala)ₐ, (Gly-Pro)ₐ, (Gly-Arg)ₐ, (Pro-Ala)ₐ and (Pro-Arg)ₐ, wherein a is an integer of 4 to 25, preferably 7 to 15, more preferably 8 to 12; and
an immunogenic carrier protein, preferably tetanus toxoid (TT), HSP60, Concholepas concholepas hemocyanin (CCH), diphtheria toxin CRM197, diphtheria toxoid (DT), meningococcal outer membrane protein complex (OMPC), ovalbumin (OVA), keyhole limpet hemocyanin (KLH) or bovine serum albumin (BSA), more preferably OVA, TT, DT and KLH,

wherein the carrier protein is non-covalently or covalently linked to the polypeptide, preferably by a spacer, more preferably by a PEG spacer. Preferred PEG spacers of the present invention have 1 to 5, more preferably 2 to 4, most preferably 3 PEG subunits.

In a preferred embodiment of the eighth aspect of the invention the carrier protein is linked to the C-terminus of the polypeptide.

In a preferred embodiment of the eighth aspect of the invention the carrier protein is linked to the N-terminus of the polypeptide.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples and figures are merely illustrative of the present invention and should not be construed to limit the scope of the invention as indicated by the appended claims in any way.

### EXAMPLES

The most promising current experimental approaches to reduce DPR expression in the brain would require regular intrathecal injection of antisense oligonucleotides (Jiang, J., et al. (2016). Neuron 90(3): 535-550; Gendron, T. F., et al. (2017). Sci Transl Med 9(383).) to achieve widespread suppression of C9orf72 repeat expression throughout the nervous system and thus are poorly suited for long-term treatment of prodromal or presymptomatic patients. Safety and efficacy have not been shown in patients so far. Using the immunogen or immunogenic composition of the present invention the inventors have shown that anti-GA vaccination can prevent motor symptoms in a poly-GA mouse model associated with reduced poly-GA aggregation and reduced neuroinflammation in the CNS. Targeting the poly-GA component may be sufficient to stop or at least significantly delay the pathogenic cascade in C9orf72 disease, because poly-GA is the most abundant DPR protein in C9orf72 patients and is most directly linked to TDP-43 pathology associated with neurodegeneration (Khosravi et al., HMG 2017; Nonaka, T., et al. (2018). Hum Mol Genet.). In addition, we show below that poly-GA causes cytoplasmic mislocalization and aggregation of TDP-43 in a non-cell autonomous fashion, which can be inhibited by monoclonal anti-GA antibodies.

### Example 1: Immunization Strategy

Wild-type (WT) and (GA)₁₄₉-CFP expressing transgenic mice (TG), as described in Schludi et al. 2017, have been immunized with aggregated (GA)₁₅ and OVA-PEG-(GA)₁₀ immunogens. The PEG spacer consisted of 3 PEG subunits. For maleimide coupling to ovalbumin a cysteinated DPR peptide (C-PEG-(GA)₁₀) was chemically synthesized (Peptide Specialty Laboratories GmbH, Heidelberg). The PEG linker was introduced during synthesis using Fmoc-8-amino-3,6-dioxaoctanoic acid. OVA conjugation was done using the Imject™ Maleimide system (Thermo Scientific). 5 mg C-PEG-(GA)₁₀ peptide dissolved in 500 μl conjugation buffer and 500 μl urea (8M, pH 7.2) were mixed with 5 mg Imject™ Maleimide-activated Ovalbumin in water (5-15 moles maleimide per mole of ovalbumin). After 6 h incubation the reaction mix was dialyzed against 400 ml PBS overnight (3500 MW cut-off). Other carrier proteins can be coupled in a similar way.

As a control group, mice were mock-immunized with PBS and adjuvant (WT-PBS or TG-PBS). Immunization followed the protocol from Mackenzie et al. 2013 with additional booster immunizations (prime-boost regimes).

For the first immunization, 40 μg OVA-PEG-(GA)₁₀ or (GA)₁₅ were mixed with 5 nmol CpG ODN 1668 oligonucleotide (Enzo Life Science) in 200 μl PBS and 250 μl incomplete Freund adjuvant and injected half-half i.p. and s.c. at 8 weeks of age. The control group (PBS) received injection of 200 μl PBS and 250 μl incomplete Freund adjuvant together with 5 nmol CpG ODN 1668.

For the five booster immunizations, 40 μg OVA-PEG-(GA)₁₀ or (GA)₁₅ were mixed with 5 nmol CpG ODN 1668 in 500 μl PBS were injected half-half i.p. and s.c. at week 12, 16, 20, 24 and 28 (see FIG. 1A).

### Example 2: Immune Response of Immunogens

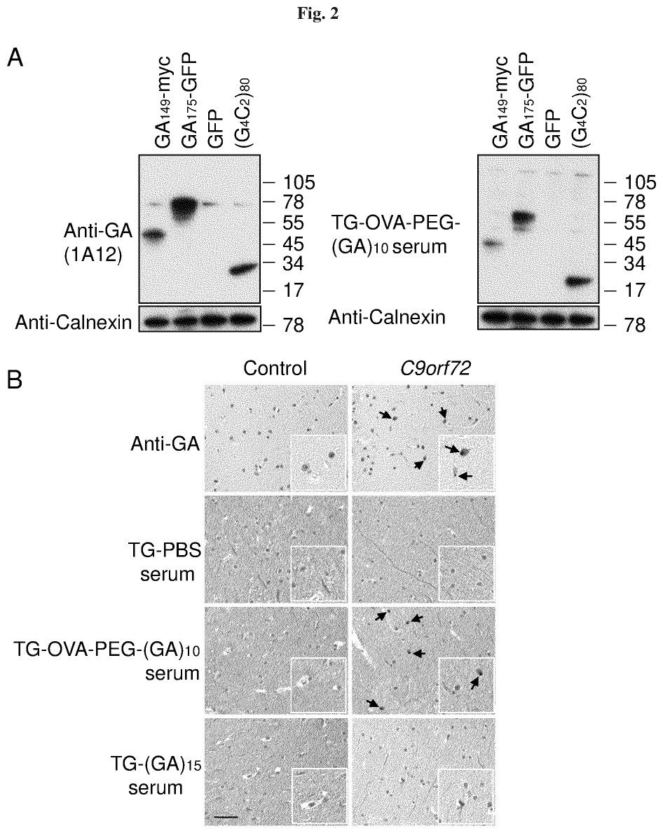

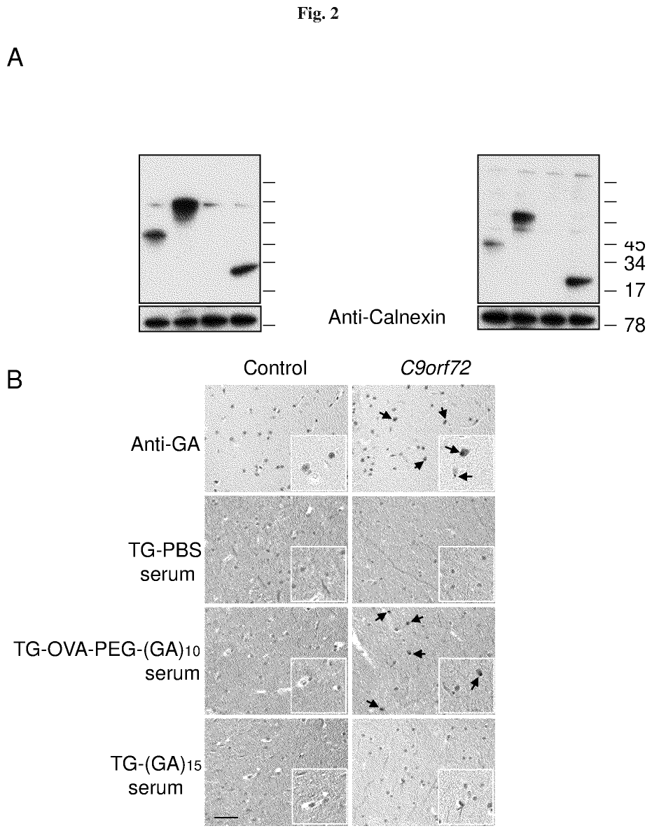

Since poly-GA is expected to be poorly immunogenic we used ovalbumin as an immunogenic carrier molecule (OVA-PEG-(GA)₁₀) that also keeps (GA)₁₀ at least partially soluble. However, the immune system is also stimulated by particulate material (similar to virus and bacteria) and thus we tested immunization with a carrier-free self-aggregating (GA)₁₅ immunogen. ELISA measurements with antisera from vaccinated mice showed that OVA-PEG-(GA)₁₀ immunization resulted in very high titers of anti-GA antibodies in WT and TG mice (see FIG. 1B), while (GA)₁₅ did not induce anti-GA antibodies, compared to the PBS control. The antisera of OVA-PEG-(GA)₁₀ immunized mice detected poly-GA expressed in HEK293 cells from ATG-initiated synthetic genes (May et al., 2014) or from an ATG-free (G₄C₂)₈₀ construct (Mori et al., EMBO Reports 2016) (FIG. 2A) similar to monoclonal anti-GA antibodies. Moreover, the OVA-PEG-(GA)₁₀ antiserum detected the characteristic dot-like inclusions in the cerebellum of C9orf72 patients (FIG. 2B). Together these data show, that poly-GA immunization requires are strongly immunogenic carrier such as Ovalbumin or KLH to generate high affinity antibodies in vivo.

### Example 3: Therapeutic Effect of Immunization

All mice were tested bi-weekly, starting at an age of 9 weeks for motor deficits in a beam-walk test. The WT mice did not show any significant changes in the time needed to cross the raised beam (58 cm length, 8 mm thickness) throughout the course of the experiment. In contrast the TG mice receiving mock immunization (TG-PBS) needed significantly more time to cross the beam starting from week 15, indicating development of motor deficits. Some mice could not cross the beam at all and dropped down (FIG. 3B).

Although the OVA-PEG-(GA)₁₀ vaccinated GA-CFP mice showed significant impairment at 17 weeks, they improved at later time points and performed close to wild-type level, suggesting that a high antibody titer is necessary for the beneficial effect of vaccination. The OVA-PEG-(GA)₁₀ vaccinated TG mice dropped down much less during the beam walk than PBS or (GA)₁₅ immunized TG animals. Thus, OVA-GA immunization is an effective treatment for GA-CFP mice.

### Example 4: Histological Effects of Immunization

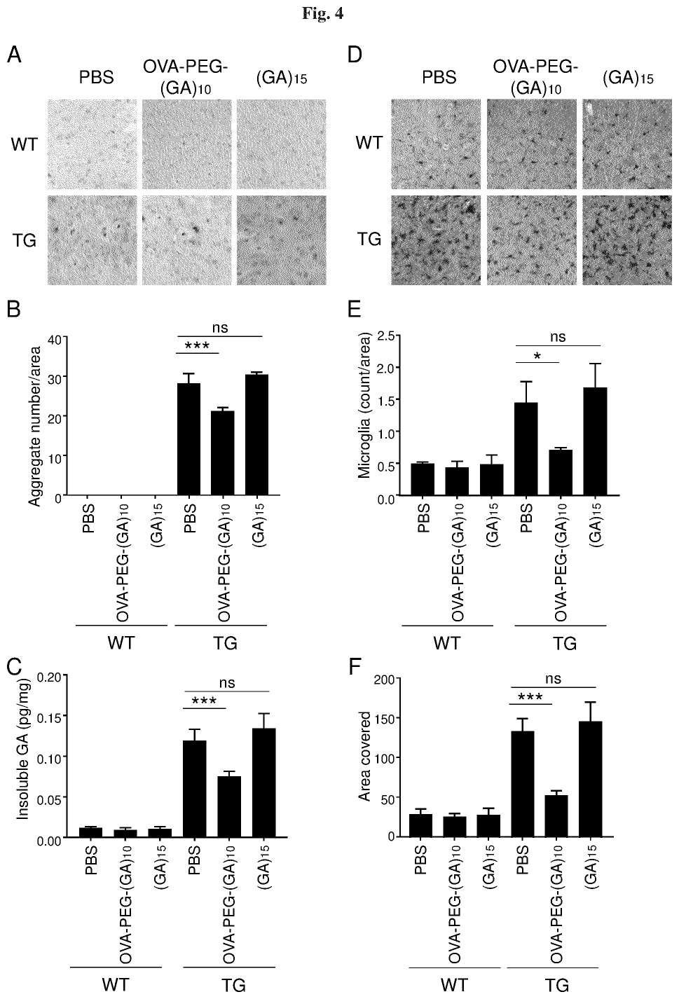

To elucidate the mode of action, we analyzed poly-GA aggregation and microglia activation in vaccinated mice after sacrificing all mice at week 32. Immunohistochemistry of the spinal cord showed the typical poly-GA aggregates in GA-CFP transgenic mice (FIG. 4A). Quantification revealed reduced poly-GA aggregate density in OVA-PEG-(GA)₁₀ vaccinated mice compared to the PBS control (FIG. 4B). This was further confirmed using a poly-GA immunoassay (FIG. 4C). Consistent with the lack of antibody response and lack of any beneficial effect, (GA)₁₅ immunization did not affect the abundance of poly-GA aggregates.

Since the reduction of poly-GA aggregates was rather modest, we asked whether antibodies may preferentially affect extracellular poly-GA and subsequent neuroinflammation. Thus, we analyzed activated microglia using Iba1 immunohistochemistry. GA-CFP transgenic mice showed strong microglia activation in the spinal cord compared to wild-type littermates (FIG. 4D). Both the density of Iba1-positive microglia and the area covered by Iba1 staining were strongly reduced in OVA-PEG-(GA)₁₀ immunized mice (FIG. 4E/F). Importantly, microglia activation in the OVA-PEG-(GA)₁₀ transgenic mice was undisguisable from wild-type mice. Taken together, OVA-PEG-(GA)₁₀ vaccination clearly reduced poly-GA aggregation and prevented microglia activation nearly completely.

Anti-GA immunoassay was performed to measure insoluble poly-GA aggregation in the spinal cord of TG mice. Mouse spinal cords were homogenized in RIPA buffer containing 137 mM NaCl, 20 mM Tris pH 7.5, 10% Glycin, 1% Triton X 100, 0.5% Na-deoxycholate, 0.1% SDS, 2 mM EDTA, protease and phosphatase inhibitors, Benzonase Nuclease. After centrifugation, pellets were resuspended, homogenized, re-centrifuged once more in RIPA buffer to avoid cross contamination between soluble and insoluble fractions. RIPA insoluble pellets were then resuspended by sonication in RIPA buffer containing 3.5M Urea (U-RIPA). ELISA was performed using biotinylated anti-GA clone 5F2 as capture antibody and anti-mouse-HRP as detection antibody.

The immunization strategy applied did not only prevent/treat motor deficits but also effectively reduce poly-GA aggregation in the spinal cord of (GA)₁₄₉-CFP expressing transgenic mice (TG) immunized with OVA-PEG-(GA)₁₀ (see FIG. 2B).

### Example 5: Safety of Immunization Strategy

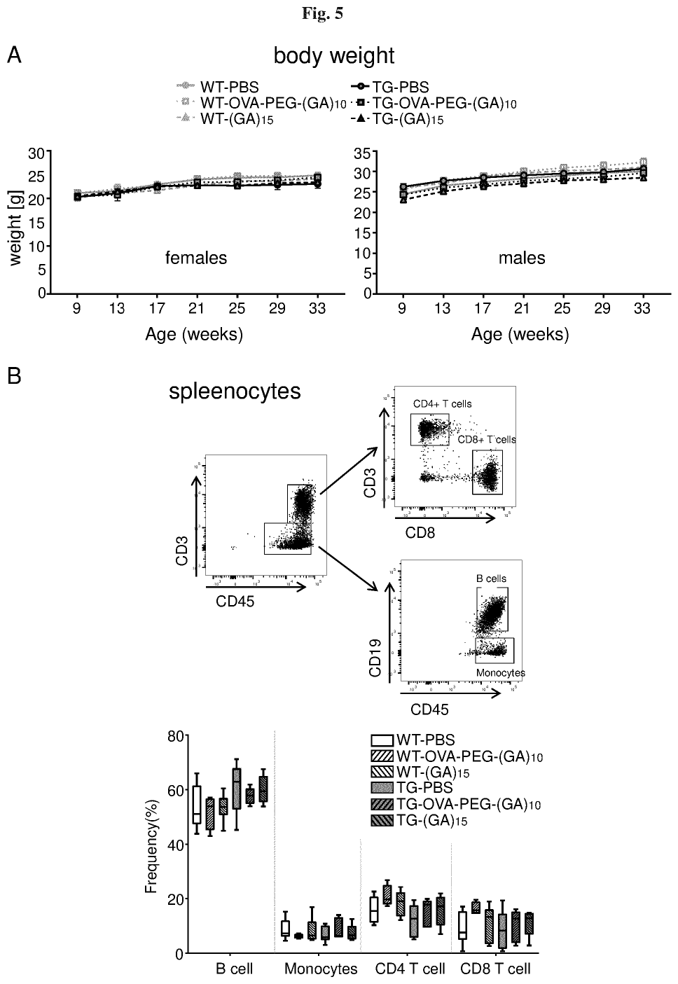

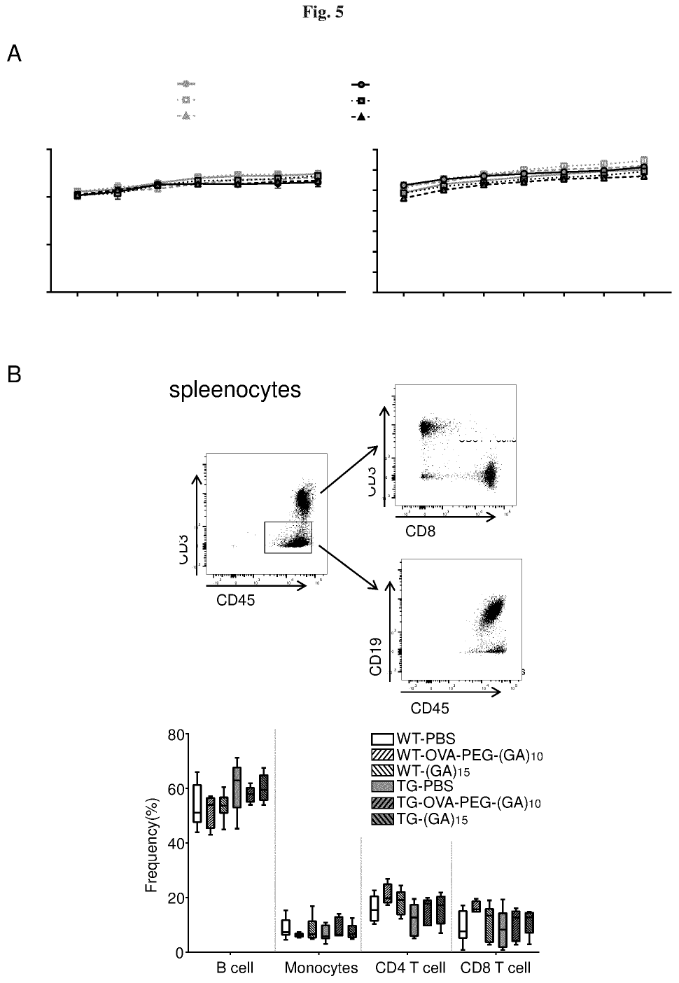

Poly-GA vaccination results in high-affinity antibodies that effectively reduce poly-GA aggregation and motor deficits with no observed side effects in mice. The immunization had no effect on body weight (FIG. 5A) and leukocyte distribution in the spleen (FIG. 5B), suggesting the boosting regimen did not alter or impair overall immune function. Poly-GA proteins are only found in C9orf72 patients and have no critical endogenous function. The human proteome does not encode for endogenous long poly-GA sequences within other proteins. Vaccination in humans will therefore be a safe and effective method to prevent or delay disease in asymptomatic mutation carriers and stop disease progression in symptomatic patients.

### Example 6: Cell-to-Cell Transmission of Poly-GA Causes Cytoplasmic Mislocalization of TDP-43

From animal experiments it is clear that toxic gain of function mechanisms of the C9orf72 repeat expansion are the main pathomechanisms and DPR proteins play the dominant role (Chew, J., et al. (2015). Science 348(6239): 1151-1154; Jiang, J., et al. (2016). Neuron 90(3): 535-550). However, neurodegeneration in C9orf72 patients correlates better with TDP-43 inclusion pathology also found in other forms of ALS/FTD than with DPR pathology, but it is not clear how the C9orf72 repeat expansion triggers TDP-43 pathology (Mackenzie et al., Acta Neuropathol 2013).

Among the DPR proteins, poly-GA has been most robustly linked to TDP-43 aggregation, although the mechanism is still unknown (Khosravi et al., 2016; Nonaka et al., 2018; Solomon et al., 2018a).

Since TDP-43 and poly-GA only occasionally co-aggregate in patient tissue, the inventors investigated potential non-cell-autonomous effects of poly-GA on TDP-43 in co-culture assays. They focused on cytoplasmic mislocalization and aggregation of TDP-43 because these are the key features of TDP-43 pathology in ALS/FTD.

Primary rat hippocampal neurons were transduced (protocol in Guo et al, Cell 2018) and grown on coverslips with either GFP or (GA)₁₇₅-GFP (“donor cells”) using lentivirus (May et al., 2014; Khosravi et al., 2017). Four days later, the coverslips were transferred with extensively washed donor cells into a new well containing untreated primary neurons (“receiver cells”) separated by −1 mm paraffin spacers (FIG. 6A) and co-cultured donor and receiver cells for another four days. Immunofluorescence of the donor cells showed enhanced cytoplasmic localization of TDP-43 in (GA)₁₇₅-GFP compared to GFP transduced donor cells (FIG. 6B/C) as the inventors had reported previously (Khosravi et al., 2016). Automated quantification of the number of poly-GA aggregates per cell (2.23±0.18 (mean±SD) in the donor compartment vs 0.70±0.22 in the receiver compartment) using ImageJ showed robust transmission of (GA)₁₇₅-GFP aggregates between neurons consistent with previous results (Westergard et al., 2016; Zhou et al., 2017). Using automated image analysis of single confocal sections with Columbus Acapella version 2.6.0, cytoplasmic TDP-43 was compared in GFP-positive and GFP-negative cells (Khosravi et al., 2016). Strikingly, cytoplasmic TDP-43 expression was not only enhanced in cells taking up visible (GA)₁₇₅-GFP aggregates but also in neurons without detectable (GA)₁₇₅-GFP, both on donor and receiver coverslips (arrowheads in FIG. 6B and quantification in FIG. 6C). Thus, poly-GA release from transduced neurons leads to TDP-43 mislocalization in neighboring neurons presumably even by uptake of small amounts of soluble or aggregated poly-GA below the detection limit.

To differentiate the effect of poly-GA on nucleocytoplasmic transport and aggregation of TDP-43, we analyzed receiver cells expressing GFP-tagged human TDP-43 lacking a functional nuclear localization signal (ΔNLS) due to K95A/K97A/R98A mutation as described before (Winton et al., 2008). Since TDP-43_{ΔNLS} is highly toxic to primary neurons as shown in mouse models (Walker et al., 2015), these co-culture experiments were conducted in HeLa cells. Donor cells were co-transfected with TDP-43_{ΔNLS}-GFP and either iRFP670 or GA₁₇₅-iRFP670 (Shcherbakova and Verkhusha, 2013), while receiver cells were transfected only with GFP-tagged TDP-43_{ΔNLS}. 24 h after separate transfection, the washed coverslips were co-cultured for another 24 h before analysis of poly-GA and TDP-43 fluorescence. Strikingly, both co-expression of poly-GA and co-culture with poly-GA expressing cells resulted in partial cytoplasmic aggregation of TDP-43_{ΔNLS}-GFP demonstrating that poly-GA has profound cell-autonomous and non-cell-autonomous effects on TDP-43 aggregation (FIG. 6D/E). In addition, the inventors confirmed that poly-GA induced TDP-43_{ΔNLS}-GFP aggregation using a filter trap assay (Mori et al., 2013) of cell extracts from the donor and receiver cells (FIG. 6F). Thus, transmission of small poly-GA amounts triggers TDP-43 mislocalization in cells without readily detectable DPR pathology.

### Example 7: Anti-GA Antibodies Block the Non-Cell-Autonomous Effects of Poly-GA on TDP-43 Mislocalization

The inventors have previously shown that monoclonal antibodies can inhibit cell-to-cell transmission of poly-GA (Zhou et al., 2017) and therefore proposed that antibodies generated due to active vaccination have a similar effect. Therefore, the inventors investigated whether anti-GA antibodies would inhibit poly-GA dependent TDP-43 mislocalization. An anti-GA antibody (1 μg/ml, clone 5F2, Zhou et al., 2017) or purified mouse IgG (1 μg/ml) as control was added to the co-culture model from FIG. 6A and TDP-43 localization was analyzed in the poly-GA transduced donor compartment and the non-transduced receiver compartment (FIG. 7). Importantly, anti-GA treatment reduced cytoplasmic TDP-43 levels in 5F2 treated neurons in the donor and especially in the receiver compartment further supporting a non-cell-autonomous role of poly-GA in TDP-43 mislocalization. Moreover, anti-GA treatment specifically reduced poly-GA levels in both compartments as shown by immunoblotting with an anti-GFP antibody (FIG. 7C). Taken together, anti-GA antibodies reduce poly-GA aggregation and transmission as well as cytoplasmic mislocalization of TDP-43.

### Example 8: Poly-GA Immunization Partially Rescue Neuroinflammatory Gene Expression Signatures

To elucidate the mode of action of active immunotherapy, the inventors performed unbiased RNA sequencing analysis on spinal cord (GA)₁₄₉-CFP expressing transgenic mice (TG) immunized with OVA-PEG-(GA)₁₀ or PBS control and their respective wild-type (WT) control mice. Pairwise comparison revealed no significant difference between the wild-type groups, but 373 differentially expressed genes between TG-PBS vs. WT-PBS and 233 between TG-OVA-PEG-(GA)₁₀ vs. TG-PBS using 1.5-fold change (log₂>0.585) as cutoff (Zhou et al, EMBO Mol Med 2020). A heatmap of expression changes is shown in FIG. 8A. 164 genes differentially expressed in TG-PBS were significantly rescued in the immunized mice (FIG. 8B). Poly-GA expression triggered many immune pathways including production of multiple cytokines/chemokines. OVA-PEG-(GA)₁₀ immunization attenuated several immune pathways, e.g. induction of Ccl4, Gm, Tyrobp and components of the complement system (C1qc, C5ar1). Gene ontology analysis shows enrichment of terms related to the immune system and cell death in both the genes rescues by OVA-PEG-(GA)₁₀ and in those genes not significantly rescued (FIG. 8C). For example, the GO term “positive regulation of cytokine production” (GO:0001819) is enriched among the significantly rescued genes (Agt, B2m, C5ar1, Ccl4, Cebpb, Clu, Csf1r, Egr1, Fcer1g, Glmn, Gpsm3, Hspb1 Icos1, Il1r1, Lgals9, Rara, Tgfb1, Tnfrsf1a, Tyrobp), but also among the non-rescued genes (Adam8, Bcl3, C3, C3ar1, Ccl2, Ccl3, Ccl5, Cd14, Cd84, Clec5a, Crlf2, Cyba, Cybb, Dhx58, Fcgr3, Fgr, Havcr2, Hpse, Il1rn, Il4ra, Irf8, Lpl, Ly9, Mmp12, Naip5, Nfam1, Pf4, Plcg2, Postn, Ptafr, Ptprc, Pycard, Runx1, Sash3, Serpinel, Slc11a1, Sulf1, Tlr1, Tlr2, Tlr7, Tlr9, Tmem173, Trem2). In contrast, the term “intrinsic apoptotic signaling pathway” (GO:0097193) is enriched only in rescued genes (Cebpb, Clu, Fbxw7, Gpx1, Hmox1, Hspb1, Nfe2l2, Pdcd10, Rnf7, Shisa5, Tnfrsf1a and Tpt1), but not in the non-rescued genes. Based on the gene ontology analysis a network of significantly rescued genes related to myeloid cells and cytokines is shown in FIG. 8D.

### Example 9: Poly-GA Immunization Reduces TDP-43 Mislocalization and Neuronal Damage

Since the present application discloses that poly-GA triggers partial cytoplasmic mislocalization of TDP-43 in a non-cell autonomous manner (FIG. 6), that can in vitro be rescued by monoclonal anti-GA antibodies (FIG. 7), the inventors also analyzed the effect of anti-GA immunization (protocol as in FIG. 1A) on the levels of cytoplasmic TDP-43 in the spinal cord (FIG. 9A/B). While (GA)₁₄₉-CFP expressing transgenic mice (TG) receiving just PBS showed a significant increase in cells with cytoplasmic TDP-43 compared to wildtype animals, transgenic mice immunized with OVA-PEG-(GA)₁₀ but not (GA)₁₅ showed reduced levels of cytoplasmic TDP-43 compared to the TG-PBS group suggesting the anti-GA antibodies induced by immunization reduce secondary TDP-43 pathology, which is an important correlate of neurodegeneration in sporadic ALS/FTD and also C9orf72 ALS/FTD (Mackenzie et al, Acta Neuropathol 2013).

Moreover, the inventors analyzed the level of neurofilament light chain (NFL), which is as a biomarker for neuroaxonal damage in ALS and other diseases (Feneberg et al, 2018; Khalil et al, 2018; Meeter et al, 2016). Indeed, a significantly reduced level of NFL in CSF of (GA)₁₄₉-CFP transgenic mice immunized with OVA-PEG-(GA)₁₀ was observed suggesting the attenuation of neuroaxonal damage in these mice, but not (GA)₁₅ immunized mice (FIG. 9C). Taken together, OVA-PEG-(GA)₁₀ vaccination partially prevented neurodegeneration and reducing TDP-43 mislocalization in poly-GA mice.

### Example 10 KLH-PEG-(GA)₁₀ and OVA-PEG-(GA)₁₀ Induce a Similar Beneficial Response

To compare the effects of different adjuvants the inventors immunized GA-CFP mice immunized with OVA-PEG-(GA)₁₀ or KLH-PEG-(GA)₁₀ starting around the known onset of motor symptoms at week 17 (compare FIG. 3A). After six monthly immunizations (FIG. 10A) OVA- and KLH-conjugated antigens induce comparable anti-GA titers (FIG. 10B). KLH conjugation is well established for immunization in humans. After the study, cerebrospinal fluid was collected from all mice at week 40 to analyze anti-GA antibodies in the CNS and a biomarker for neurodegeneration. Anti-GA antibodies were clearly detected in the CSF of OVA-PEG-(GA)₁₀ or KLH-PEG-(GA)₁₀ (median in WT OVA-PEG-(GA)₁₀ 0.9063 μg/ml, WT KLH-PEG-(GA)₁₀ 0.0541 μg/ml, TG OVA-PEG-(GA)₁₀ 0.2809 μg/ml and TG KLH-PEG-(GA)₁₀ 0.1291 μg/ml; median of all samples in these groups 0.2219 μg/ml) showing successful antibody delivery to the CNS (FIG. 10C). Importantly, both regimens reduced the levels of neurofilament light chain (NFL) levels in cerebrospinal fluid to a similar degree as when vaccination was started presymptomatically (compare FIGS. 9C and 10D).

In summary, the experiments disclosed herein show that OVA-PEG-(GA)₁₀ and KLH-PEG-(GA)₁₀ immunization is effective in the (GA)₁₄₉-CFP mouse model and does not cause overt side effects as indicated by normal weight curves and normal leukocyte profiles in the blood (FIG. 5).

The in vivo efficacy of a DPR-targeting immunization strategy had previously not been disclosed by us or others. In addition to reducing poly-GA levels and neuroinflammation, the immunization strategy disclosed herein also has positive in vivo effects on downstream TDP-43 pathology (FIG. 9A/B), which we also first disclosed herein for the treatment with a monoclonal anti-GA antibody in cellular models (FIG. 7). A recent study with two monoclonal anti-GA antibodies in a C9orf72 BAC models strikingly shows that only one of the two antibodies extends survival and efficacy cannot be predicted by affinity and other in vitro data (Nguyen et al 2020, Neuron 105: 645-662). Our data show for the first time, that an active anti-GA vaccination approach results in a surprisingly high antibody titer in the plasma and significant titers in the CSF despite predicted low immunogenicity of the hydrophobic antigen. Furthermore disclosed are beneficial effects on NFL levels, which is the best currently available fluid biomarkers for neurodegeneration. To the best of the inventors' knowledge this is the first time that an active anti-GA immunization was shown to have a therapeutic effect in an animal model of C9orf72 ALS/FTD.

### REFERENCES

DeJesus-Hernandez, M. et al., (2011). “Expanded GGGGCC hexanucleotide repeat in noncoding region of C9ORF72 causes chromosome 9p-linked FTD and ALS.” Neuron 72(2): 245-256.
Edbauer, D. and C. Haass (2016). “An amyloid-like cascade hypothesis for C9orf72 ALS/FTD.” Curr Opin Neurobiol 36: 99-106.
Gendron, T. F., et al., (2017). “Poly(GP) proteins are a useful pharmacodynamic marker for C9ORF72-associated amyotrophic lateral sclerosis.” Sci Transl Med 9(383).
Guo, Q., et al., (2018). “In Situ Structure of Neuronal C9orf72 Poly-GA Aggregates Reveals Proteasome Recruitment.” Cell 172(4): 696-705 e612.
Jiang, J., et al., (2016). “Gain of Toxicity from ALS/FTD-Linked Repeat Expansions in C9ORF72 Is Alleviated by Antisense Oligonucleotides Targeting GGGGCC-Containing RNAs.” Neuron 90(3): 535-550.
Khosravi, B., et al., (2016). “Cytoplasmic poly-GA aggregates impair nuclear import of TDP-43 in C9orf72 ALS/FTLD.” Hum Mol Genet. 2017 Feb. 15; 26(4):790-800
Mackenzie, I. R., et al., (2013). “Dipeptide repeat protein pathology in C9ORF72 mutation cases: clinico-pathological correlations.” Acta Neuropathol 126(6): 859-879.
May, S., et al., (2014). “C9orf72 FTLD/ALS-associated Gly-Ala dipeptide repeat proteins cause neuronal toxicity and Unc119 sequestration.” Acta Neuropathol 128(4): 485-503.
Mori, K., et al., (2013). “Bidirectional transcripts of the expanded C9orf72 hexanucleotide repeat are translated into aggregating dipeptide repeat proteins.” Acta Neuropathol 126(6): 881-893.
Mori, K., et al., (2013). “The C9orf72 GGGGCC repeat is translated into aggregating dipeptide-repeat proteins in FTLD/ALS.” Science 339(6125): 1335-1338.
Nonaka, T., et al., (2018). “C9ORF72 dipeptide repeat poly-GA inclusions promote: intracellular aggregation of phosphorylated TDP-43.” Hum Mol Genet. 2018 May 10 Renton, A. E., et al., (2011). “A hexanucleotide repeat expansion in C9ORF72 is the cause of chromosome 9p21-linked ALS-FTD.” Neuron 72(2): 257-268.
Rohrer, J. D., et al., (2015). “Presymptomatic cognitive and neuroanatomical changes in genetic frontotemporal dementia in the Genetic Frontotemporal dementia Initiative (GENFI) study: a cross-sectional analysis.” Lancet Neurol 14(3): 253-262.
Schludi, M. H., et al., (2017). “Spinal poly-GA inclusions in a C9orf72 mouse model trigger motor deficits and inflammation without neuron loss.” Acta Neuropathol. 2017 August; 134(2):241-254
Schludi, M. H., et al., (2015). “Distribution of dipeptide repeat proteins in cellular models and C9orf72 mutation cases suggests link to transcriptional silencing.” Acta Neuropathol 130(4): 537-555.
Simone, R., et al., (2018). “G-quadruplex-binding small molecules ameliorate C9orf72 FTD/ALS pathology in vitro and in vivo.” EMBO Mol Med 10(1): 22-31.
Su, Z., et al., (2014). “Discovery of a biomarker and lead small molecules to target r(GGGGCC)-associated defects in c9FTD/ALS.” Neuron 83(5): 1043-1050.
Vatsavayai, S. C., et al., (2016). “Timing and significance of pathological features in C9orf72 expansion-associated frontotemporal dementia.” Brain 139(Pt 12): 3202-3216.
Zhang, Y. J., et al., (2016). “C9ORF72 poly(GA) aggregates sequester and impair HR23 and nucleocytoplasmic transport proteins.” Nat Neurosci 19(5): 668-677.
Zhou, Q., et al., (2017). “Antibodies inhibit transmission and aggregation of C9orf72 poly-GA dipeptide repeat proteins.” EMBO Mol Med. 2017 May; 9(5):687-702

1. A method of treating or preventing familial frontotemporal dementia (FTD), amyotrophic lateral sclerosis (ALS) and/or amyotrophic lateral sclerosis-frontotemporal dementia (ALS-FTD), comprising administering to a subject with C9orf72 repeat expansion an immunogen comprising a polypeptide consisting of dipeptide-repeats with a sequence selected from the group consisting of (Gly-Ala)ₐ, (Gly-Pro)ₐ, (Gly-Arg)ₐ, (Pro-Ala)ₐ and (Pro-Arg)ₐ, wherein a is an integer of 4 to 25.

2. The method according to claim 1, wherein the polypeptide consists of (Gly-Ala)ₐ, wherein a is an integer of 4 to 25.

3. The method according to claim 1, wherein the immunogen further comprises an immunogenic carrier protein.

4. The method according to claim 3, wherein the carrier protein is non-covalently or covalently linked to the polypeptide.

5. The method according to claim 1, wherein the FTD and ALS is caused by the expansion of a (GGGGCC)ₙ hexanucleotide repeat upstream of C9orf72 coding region.

6. The method according to claim 1, wherein the subject is presymptomatic or prodromal.

7. The method according to claim 1, wherein the administering comprises a prime-boost regimen, comprising at least one boost application, wherein optionally the prime-boost regimen is followed by a continuous boost regimen consisting of life-long boost applications.

8. An immunogenic composition comprising
(a) the immunogen of claim 1, and
(b) a pharmaceutically acceptable carrier and/or suitable excipient(s).

9. The immunogenic composition according to claim 8 further comprising an adjuvant.

10. A method of treating or preventing of FTD, ALS and/or ALS-FTD in a subject, the method comprising administering to the subject an immunogenic composition according to claim 1 and further comprising a pharmaceutically acceptable carrier and/or suitable excipient(s).

11. A kit comprising the immunogenic composition according to claim 8 and instructions for use in prevention or treatment of FTD, ALS and/or ALS-FTD.

12. (canceled)

13. A nucleic acid encoding the immunogen according to claim 1.

14. A vector comprising the nucleic acid of claim 13.

15. An immunogen comprising
a. a polypeptide comprising dipeptide-repeats with a sequence selected from the group consisting of (Gly-Ala)ₐ, (Gly-Pro)ₐ, (Gly-Arg)ₐ, (Pro-Ala)ₐ and (Pro-Arg)ₐ, wherein a is an integer of 4 to 25; and
b. an immunogenic carrier protein,
wherein the carrier protein is non-covalently or covalently linked to the polypeptide.

16. The method according to claim 3, wherein the immunogenic carrier protein is selected from tetanus toxoid (TT), HSP60, Concholepas concholepas hemocyanin (CCH), diphtheria toxin CRM197, diphtheria toxoid (DT), meningococcal outer membrane protein complex (OMPC), ovalbumin (OVA), keyhole limpet hemocyanin (KLH) and bovine serum albumin (BSA).

17. The method according to claim 16, wherein the immunogenic carrier protein is selected from OVA, TT, DT and KLH.

18. The method according to claim 4, wherein the carrier protein is covalently linked to the polypeptide by a spacer.

19. The immunogenic composition according to claim 8, further comprising an adjuvant selected from:
a. incomplete Freund adjuvant
b. CpG oligodeoxynucleotides,
c. Hiltonol (Poly-ICLC),
d. inorganic adjuvants;
e. organic adjuvants;
f. oil in water emulsions;
g. cytokines;
h. particulate adjuvants;
i. virosomes;
j. bacterial adjuvants;
k. synthetic adjuvants; and
l. combinations thereof.

20. The vector according to claim 14, wherein the vector is a viral vector.
